(19) United States
(12) Patent Application Publication
(10) Pub. No.: US 2026/0158103 A1
(43) Pub. Date: Jun. 11, 2026

(54) USE OF CYCLOSPORINE ANALOGUES AS ANTITHROMBOTIC AGENTS

(71) Applicant: Hepion Pharmaceuticals, Inc., Edison, NJ (US)

(72) Inventors: Daren R. Ure, Edmonton (CA); Robert T. Foster, Edmonton (CA); Daniel J. Trepanier, Edmonton (CA); Patrick R. Mayo, Edmonton (CA)

(21) Appl. No.: 19/305,421

(22) Filed: Aug. 20, 2025

Related U.S. Application Data
(63) Continuation of application No. 17/535,692, filed on Nov. 26, 2021, now abandoned.
(60) Provisional application No. 63/118,947, filed on Nov. 29, 2020.

(51) Int. Cl. A61K 38/13; A61K 45/06; A61P 7/02

(52) U.S. Cl. CPC A61K 38/13; A61K 45/06; A61P 7/02

(57) ABSTRACT

Disclosed herein include methods, compositions, and kits suitable for use in preventing/treating a thromboembolic disorder, preventing/reducing formation of thrombi, and reducing/inhibiting procoagulant platelet formation. The methods comprise administering to a subject in need thereof a composition comprising a cyclosporine analogue (for example, CRV431). The compositions and kits comprise a cyclosporine analogue.

### CROSS-REFERENCE TO RELATED APPLICATIONS

This application claims the benefit of and priority to U.S. patent application Ser. No. 17/535,692 filed Nov. 26, 2021 which claims priority to U.S. Prov. Appl. No. 63/118,947, filed on Nov. 29, 2020, the content of each of which is incorporated herein by reference in its entirety.

### BACKGROUND

### Field

The present disclosure relates generally to the fields of molecular biology and medicine. One aspect relates to reducing formation of blood clots (thrombi) with cyclophilin inhibitors.

### Description of the Related Art

Thrombosis occurs in response to injury or other signal that leads to the recruitment of platelets to the injury site. The platelets adhere to the injury site to form a plug and are major constituents of clots. In addition, coagulation factors are released that further activate platelets and generate the protein fibrin, another major component of clots. In the coagulation pathway, the extrinsic pathway is activated by external injury, while the intrinsic pathway is activated by trauma within the vascular system. Both of these converge on the common pathway to promote clotting.

Three classes of drugs are now used for thrombosis treatments. Two major ones are anti-coagulants and anti-platelet agents. The third class, thrombolytics, target fibrin and are used to dissolve clots. Thrombolytics are typically used in emergency situations (for example, stroke), and even then, are used rarely as they can induce severe bleeding and even cerebral hemorrhage. Anti-coagulant therapies inhibit the proteolytic signaling pathways that induce fibrin production and promote platelet activation. These anti-coagulant therapies include drugs such as warfarin, which targets the intrinsic pathway of the coagulation response. Anti-platelet therapies can block platelet activation and aggregation. Anti-platelet therapies include aspirin, which inhibits an enzyme critical for platelet activation. Many of these drugs can be associated with dangerous bleeding.

There is a need to find antithrombotic agents that are effective in preventing clotting and thrombi, but are not as likely to cause bleeding as current therapies do.

### SUMMARY

Disclosed herein include methods of reducing or preventing formation of blood clots (thrombi), methods of treating a thromboembolic disorder, methods of primary prophylaxis or secondary prophylaxis of a thromboembolic disorder, and methods of reducing or inhibiting procoagulant platelet formation. In some embodiments, the methods comprise, administering to a subject in need thereof a composition comprising a cyclosporine analogue of Formula L, or a pharmaceutically acceptable salt, solvate, or stereoisomer thereof, thereby reducing the formation of blood clots in the subject:

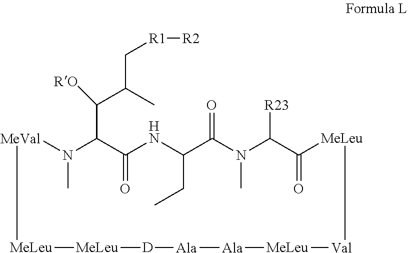

wherein:
a. R′ is H or acetyl;
b. R1 is a saturated or unsaturated straight or branched aliphatic carbon chain from 2 to 15 carbon atoms in length;
c. R2 is selected from the group consisting of:
   i. H;
   ii. an unsubstituted, N-substituted, or N,N-disubstituted amide;
   iii. a N-substituted or unsubstituted acyl protected amine;
   iv. a N-substituted or unsubstituted amine;
   v. a carboxylic acid;
   vi. a nitrile;
   vii. an ester;
   viii. a ketone,
   ix. a hydroxy, dihydroxy, trihydroxy, or polyhydroxy alkyl; and
   x. a substituted or unsubstituted aryl;
   xi. a saturated or unsaturated, straight or branched aliphatic chain optionally containing a substituent selected from the group consisting of a hydrogen, a ketone, a hydroxyl, a nitrile, a carboxylic acid, an ester, a 1,3-dioxolane, a halogen, and an oxo;
   xii. an aromatic group containing a substituent selected from the group consisting of a halogen, an ester, and a nitro; and
   xiii. a combination of the saturated or unsaturated, straight or branched aliphatic chain of (xi) and the aromatic group of (xii); and
d. R23 is a saturated or unsaturated straight chain or branched optionally substituted aliphatic carbon chain.

In some embodiments, the cyclosporine analogue of Formula L is CRV431

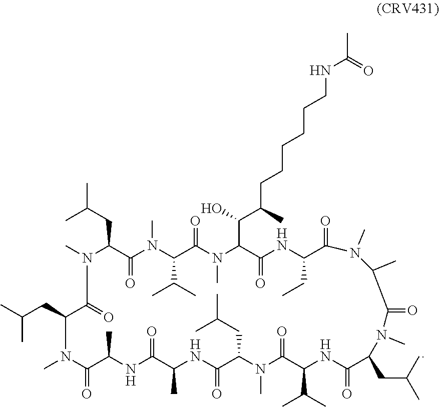 [SMILES: CC[C@@H]1NC(=O)C([C@H](O)[C@H](C)CCCCCCNC(C)=O)N(C)C(=O)[C@H](C(C)C)N(C)C(=O)[C@H](CC(C)C)N(C)C(=O)[C@H](CC(C)C)N(C)C(=O)[C@@H](C)NC(=O)[C@H](C)NC(=O)[C@H](CC(C)C)N(C)C(=O)[C@H](C(C)C)NC(=O)[C@H](CC(C)C)N(C)C(=O)C(C)N(C)C1=O]

in some embodiments, the composition comprises a therapeutically or prophylactically effective amount of the cyclosporine analogue (e.g., CRV431). The thromboembolic disorder can be, for example, an arterial cardiovascular thromboembolic disorder, a venous cardiovascular thromboembolic disorder, or a thromboembolic disorder in a chamber of the heart or in the peripheral circulation. The thromboembolic disorder is, in some embodiments, unstable angina, an acute coronary syndrome, myocardial infarction, transient ischemic attack, stroke, atherosclerosis, peripheral occlusive arterial disease, venous thrombosis, deep vein thrombosis, thrombophlebitis, arterial embolism, coronary arterial thrombosis, cerebral arterial thrombosis, cerebral embolism, kidney embolism, pulmonary embolism, or thrombosis resulting from a medical implant, device, or procedure in which blood is exposed to an artificial surface that promotes thrombosis. In some embodiments, the thromboembolic disorder is stroke, myocardial infarction, unstable angina, abrupt closure following angioplasty or stent placement, thrombosis induced by peripheral vascular surgery or peripheral vascular disease, or a thrombotic disorder resulting from atrial fibrillation or inflammation.

The method, in some embodiments, comprises reducing thrombi formation in the subject by at least 5%, 10%, 20%, 50%, 70%, 90%, or more. The method, in some embodiments, comprises delaying or reducing the likelihood of thrombi formation in the subject. For example, the method can delay the likelihood of thrombi formation in the subject by, or by at least, one minute, two minutes, three minutes, five minutes, 10 minutes, 20 minutes, 30 minutes, 45 minutes, one hour, two hours, three hours, four hours, five hours, or more, or a range between any of these values. In some embodiments, the method reduces the likelihood of thrombi formation in the subject by, or by at least, 5%, 10%, 20%, 30%, 40%, 50%, 60%, 70%, 80%, 90%, 95%, or more, or a range between any two of these values. The method, in some embodiments, comprises preventing or delaying the onset of thrombi formation in the subject. For example, the method can delay the onset of thrombi formation in the subject by, or by at least, one minute, two minutes, three minutes, five minutes, 10 minutes, 20 minutes, 30 minutes, 45 minutes, one hour, two hours, three hours, four hours, five hours, or more, or a range between any of these values. The method, in some embodiments, comprises reducing the bleeding risk in the subject by at least 5%, 10%, 20%, 50%, 70%, 90%, or more. In some embodiments, platelet aggregation in the subject is not inhibited. In some embodiments, platelet aggregation in the subject is reduced by no more than 80%, 70%, 60%, 50%, 40%, 30%, 20%, 10%, or 5% in the subject as compared to an untreated subject. In some embodiments, the cyclosporine analogue selectively inhibits formation of procoagulant platelets. The method, in some embodiments, comprises selectively inhibiting formation of procoagulant platelets. The method, in some embodiments, reduces thrombi formation without inhibiting platelet aggregation. The method, in some embodiments, does not inhibit platelet aggregation.

In some embodiments, the subject in need thereof is a subject suffering from or at risk of developing a prothrombotic or thrombotic condition. Non-limiting examples of the prothrombotic and thrombotic condition include infection, sepsis, systemic inflammatory response syndrome, multi organ failure, thrombotic thrombocytopenia purpura, hemolytic uremia syndrome, vascularization, renal failure, ischemic repercussion injury, solid organ transplant rejection, cardiovascular disease, stroke, venous thromboembolism, autoimmune disorders, sickle cell disease, inflammatory bowel disease, acute lung injury, malignancy, myocardial infarction (primary and secondary), embolic stroke, ischemic stroke, thrombotic stroke, deep vein thrombosis (DVT), thromboembolism, portal vein thrombosis, renal vein thrombosis, jugular vein thrombosis, Budd-Chiari syndrome, Paget-Schroetter disease, cerebral venous sinus thrombosis, arterial thrombosis, and arterial embolism.

In some embodiments, the composition comprises a therapeutically or prophylactically effective amount of the cyclosporine analogue (e.g., CRV431). In some embodiments, the subject is a mammal, for example a human. The human can be, for example, an elderly (e.g., a human at least 60 years old) or a minor (e.g., a human no more than 18 years old). In some embodiments, the composition comprises one of more pharmaceutically acceptable excipients. In some embodiments, the composition comprises one or more additional therapeutic agents. In some embodiments, the method further comprises administering to the subject in need thereof one or more additional therapeutic agents. The one or more additional therapeutic agents can comprise, for example, an anti-coagulant agent, an antiplatelet agent, a fibrinolytic agent, or a combination thereof. Non-limiting examples of the additional therapeutic agent include heparin, low molecular weight heparins, bivalirudin, Fondaparinux, warfarin, Acenocoumarol, Phenprocoumon, Phenindione. Abbokinase (urokinase), streptokinase, alteplase, retaplase, tenecteplase, prasugrel, aspirin, ticlopidine, clopidogrel, abciximab, eptifibatide, tirofiba, or a combination thereof. In some embodiments, at least one of the one or more additional therapeutic agents is co-administered to the subject with the composition. In some embodiments, at least one of the one or more additional therapeutic agents is administered to the subject before the administration of the composition, after the administration of the composition, or both. In some embodiments, the composition is administered to the subject by intravenous administration, oral administration, or parenteral administration, for example by oral or intravenous administration. In some embodiments, the composition is in the form of powder, pill, tablet, microtablet, pellet, micropellet, capsule, capsule containing microtablets, liquid, aerosols, or nanoparticles. In some embodiments, the composition is administered to the subject at an effective daily dose of the cyclosporine analogue or a pharmaceutically acceptable salt, solvate, or stereoisomer thereof at from 10 mg to 250 mg.

Also disclosed herein include a kit, comprising a cyclosporine analogue of Formula L, or a pharmaceutically acceptable salt, solvate, stereoisomer, or composition thereof, and a label indicating the use of the kit. In some embodiments, the label indicates that the kit is for preventing or treating a thromboembolic disorder. In some embodiments, the label indicates that the kit is for preventing or reducing thrombi formation. In some embodiments, the label indicates that the kit is for reducing or inhibiting procoagulant platelet formation. In some embodiments, the kit comprises a label indicating that the kit is for preventing or treating a thromboembolic disorder. In some embodiments, the kit comprises a label indicating that the kit is for reducing or inhibiting procoagulant platelet formation.

In some embodiments, the thromboembolic disorder is an arterial cardiovascular thromboembolic disorder, a venous cardiovascular thromboembolic disorder, or a thromboembolic disorder in a chamber of the heart or in the peripheral circulation. The thromboembolic disorder can be unstable angina, an acute coronary syndrome, myocardial infarction, transient ischemic attack, stroke, atherosclerosis, peripheral occlusive arterial disease, venous thrombosis, deep vein thrombosis, thrombophlebitis, arterial embolism, coronary arterial thrombosis, cerebral arterial thrombosis, cerebral embolism, kidney embolism, pulmonary embolism, or thrombosis resulting from a medical implant, device, or procedure in which blood is exposed to an artificial surface that promotes thrombosis. The thromboembolic disorder can be stroke, myocardial infarction, unstable angina, abrupt closure following angioplasty or stent placement, thrombosis induced by a peripheral vascular surgery or a peripheral vascular disease, or a thrombotic disorder resulting from atrial fibrillation or inflammation.

In some embodiments, the subject suffers from or is at risk of developing a prothrombotic or thrombotic condition. The prothrombotic or thrombotic condition can be selected from the group consisting of infection, sepsis, systemic inflammatory response syndrome, multi organ failure, thrombotic thrombocytopenia purpura, hemolytic uremia syndrome, vascularization, renal failure, ischemic repercussion injury, solid organ transplant rejection, cardiovascular disease, stroke, venous thromboembolism, autoimmune disorders, sickle cell disease, inflammatory bowel disease, acute lung injury, malignancy, myocardial infarction (primary and secondary), embolic stroke, ischemic stroke, thrombotic stroke, deep vein thrombosis (DVT), thromboembolism, portal vein thrombosis, renal vein thrombosis, jugular vein thrombosis, Budd-Chiari syndrome, Paget-Schroetter disease, cerebral venous sinus thrombosis, arterial thrombosis, and arterial embolism.

In some embodiments, the cyclosporine analogue is CRV431. In some embodiments, the label comprises instructions to administer a therapeutically or prophylactically effective amount of the cyclosporine analogue. In some embodiments, the subject is a mammal, such as a human. In some embodiments, the composition of cyclosporine comprises one or more pharmaceutically acceptable excipients.

In some embodiments, the label comprises instructions to administer the cyclosporine analogue with one or more additional therapeutic agents. The kit can further comprise one or more of the one or more additional therapeutic agents. The one or more additional therapeutic agents can comprise an anti-coagulant agent, an antiplatelet agent, a fibrinolytic agent, or a combination thereof. The one or more additional therapeutic agents can comprise heparin, low molecular weight heparins, bivalirudin, Fondaparinux, warfarin, Acenocoumarol, Phenprocoumon, Phenindione, Abbokinase (urokinase), streptokinase, alteplase, retaplase, tenecteplase, prasugrel, aspirin, ticlopidine, clopidogrel, abciximab, eptifibatide, tirofiba, or a combination thereof. In some embodiments, the instructions comprise instructions to co-administer at least one of the one or more additional therapeutic agents with the cyclosporine analogue to the subject. In some embodiments, the instructions comprise instructions to administer at least one of the one or more additional therapeutic agents to the subject before the administration of the cyclosporine analogue to the subject, after the administration of the cyclosporine analogue to the subject, or both.

In some embodiments, the instructions comprise instructions to administer the cyclosporine analogue to the subject by intravenous administration, oral administration, or parenteral administration. The instructions can comprise instructions to administer the cyclosporine analogue to the subject by oral or intravenous administration. In some embodiments, the instructions comprise instructions to administer the cyclosporine analogue to the subject at an effective daily dose of the cyclosporine analogue or a pharmaceutically acceptable salt, solvate, or stereoisomer thereof, at from 10 mg to 250 mg. In some embodiments, the cyclosporine analogue is in the form of powder, pill, tablet, microtablet, pellet, micropellet, capsule, capsule containing microtablets, liquid, stable self-microemulsifying drug delivery system (SMEDD), aerosols, or nanoparticles.

Disclosed herein include a pharmaceutically composition comprising a cyclosporine analogue of Formula L, or a pharmaceutically acceptable salt, solvate, or stereoisomer thereof, for use in treating a thromboembolic disorder; a pharmaceutically composition comprising a cyclosporine analogue of Formula L, or a pharmaceutically acceptable salt, solvate, or stereoisomer thereof, for use in preventing or reducing formation of thrombi, and a pharmaceutically composition comprising a cyclosporine analogue of Formula L, or a pharmaceutically acceptable salt, solvate, or stereoisomer thereof, for use in reducing or inhibiting procoagulant platelet formation. The pharmaceutical composition can be for administration to a subject, e.g., a mammal such as a human. In some embodiments, the cyclosporine analogue is CRV431.

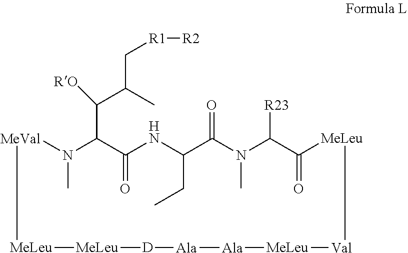

wherein:
a. R′ is H or acetyl;
b. R1 is a saturated or unsaturated straight or branched aliphatic carbon chain from 2 to 15 carbon atoms in length;
c. R2 is selected from the group consisting of:
   i. H;
   ii. an unsubstituted, N-substituted, or N,N-disubstituted amide;
   iii. a N-substituted or unsubstituted acyl protected amine;
   iv. a N-substituted or unsubstituted amine;
   v. a carboxylic acid;
   vi. a nitrite;
   vii. an ester;
   viii. a ketone;
   ix. a hydroxy, dihydroxy, trihydroxy, or polyhydroxy alkyl; and
   x. a substituted or unsubstituted aryl;
   xi. a saturated or unsaturated, straight or branched aliphatic chain optionally containing a substituent selected from the group consisting of a hydrogen, a ketone, a hydroxyl, a nitrile, a carboxylic acid, an ester, a 1,3-dioxolane, a halogen, and an oxo;
   xii. an aromatic group containing a substituent selected from the group consisting of a halogen, an ester, and a nitro; and
   xiii. a combination of the saturated or unsaturated, straight or branched aliphatic chain of (xi) and the aromatic group of (xii); and
d. R23 is a saturated or unsaturated straight chain or branched optionally substituted aliphatic carbon chain.

In some embodiments, the thromboembolic disorder is an arterial cardiovascular thromboembolic disorder, a venous cardiovascular thromboembolic disorder, or a thromboembolic disorder in a chamber of the heart or in the peripheral circulation. In some embodiments, the thromboembolic disorder is unstable angina, an acute coronary syndrome, myocardial infarction, transient ischemic attack, stroke, atherosclerosis, peripheral occlusive arterial disease, venous thrombosis, deep vein thrombosis, thrombophlebitis, arterial embolism, coronary arterial thrombosis, cerebral arterial thrombosis, cerebral embolism, kidney embolism, pulmonary embolism, or thrombosis resulting from a medical implant, device, or procedure in which blood is exposed to an artificial surface that promotes thrombosis. In some embodiments, the thromboembolic disorder is stroke, nocardial infarction, unstable angina, abrupt closure following angioplasty or stent placement, thrombosis induced by peripheral vascular surgery or peripheral vascular disease, or a thrombotic disorder resulting from atrial fibrillation or inflammation.

In some embodiments, the subject suffers from or is at risk of developing a prothrombotic or thrombotic condition. The prothrombotic or thrombotic condition can be selected from the group consisting of infection, sepsis, systemic inflammatory response syndrome, multi organ failure, thrombotic thrombocytopenia purpura, hemolytic uremia syndrome, vascularization, renal failure, ischemic repercussion injury, solid organ transplant rejection, cardiovascular disease, stroke, venous thromboembolism, autoimmune disorders, sickle cell disease, inflammatory bowel disease, acute lung injury, malignancy, myocardial infarction (primary and secondary), embolic stroke, ischemic stroke, thrombotic stroke, deep vein thrombosis (DVT), thromboembolism, portal vein thrombosis, renal vein thrombosis, jugular vein thrombosis, Budd-Chiari syndrome, Paget-Schroetter disease, cerebral venous sinus thrombosis, arterial thrombosis, and arterial embolism.

In some embodiments, the pharmaceutical composition comprises one or more pharmaceutically acceptable excipients. In some embodiments, the pharmaceutical composition is for administration with one or more additional therapeutic agents. The pharmaceutical composition can further comprise one or more of the one or more additional therapeutic agents. The one or more additional therapeutic agents can comprise an anti-coagulant agent, an antiplatelet agent, a fibrinolytic agent, or a combination thereof. The one or more additional therapeutic agents can comprise heparin, low molecular weight heparins, bivalirudin, Fondaparinux, warfarin, Acenocoumarol, Phenprocoumon, Phenindione, Abbokinase (urokinase), streptokinase, alteplase, retaplase, tenecteplase, prasugrel, aspirin, ticlopidine, clopidogrel, abciximab, eptifibatide, tirofiba, or a combination thereof.

In some embodiments, the at least one of the one or more additional therapeutic agents is for co-administration with the cyclosporine analogue to the subject. In some embodiments, at least one of the one or more additional therapeutic agents is for administration to the subject before the administration of the pharmaceutical composition to the subject, after the administration of the pharmaceutical composition to the subject, or both. In some embodiments, the pharmaceutically composition is formulated for intravenous administration, oral administration, or parenteral administration. The pharmaceutical composition can be formulated for oral or intravenous administration in some embodiments, the pharmaceutical composition is for administration to the subject at an effective daily dose of the cyclosporine analogue or a pharmaceutically acceptable salt, solvate, or stereoisomer thereof, at from 10 mg to 250 mg. In some embodiments, the pharmaceutically composition is in the form of powder, pill, tablet, microtablet, pellet, micropellet, capsule, capsule containing microtablets, liquid, stable self-microemulsifying drug delivery system (SMEDD), aerosols, or nanoparticles.

Disclosed herein includes embodiments of use of a pharmaceutically composition, comprising a cyclosporine analogue of Formula L, or a pharmaceutically acceptable salt, solvate, or stereoisomer thereof, for the manufacture of a medicament for treating a thromboembolic disorder, or for preventing or reducing thrombi formation, or for reducing or inhibiting procoagulant platelet formation in a subject,

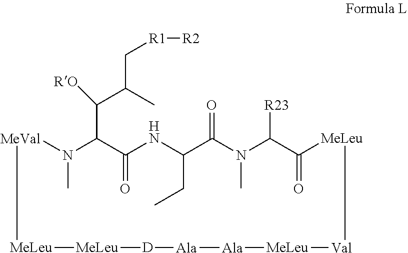

wherein:
a. R′ is H or acetyl;
b. R1 is a saturated or unsaturated straight or branched aliphatic carbon chain from 2 to 15 carbon atoms in length;
c. R2 is selected from the group consisting of:
   i. H;
   ii. an unsubstituted, N-substituted, or N,N-disubstituted amide;
   iii. a N-substituted or unsubstituted acyl protected amine;
   iv. a N-substituted or unsubstituted amine;
   v. a carboxylic acid;
   vi. a nitrile;
   vii. an ester;
   viii. a ketone;
   ix. a hydroxy, dihydroxy, trihydroxy, or polyhydroxy alkyl; and
   x. a substituted or unsubstituted aryl;
   xi. a saturated or unsaturated, straight or branched aliphatic chain optionally containing a substituent selected from the group consisting of a hydrogen, a ketone, a hydroxyl, a nitrile, a carboxylic acid, an ester, a 1,3-dioxolane, a halogen, and an oxo;
   xii. an aromatic group containing a substituent selected from the group consisting of a halogen, an ester, and a nitro; and
   xiii. a combination of the saturated or unsaturated, straight or branched aliphatic chain of (xi) and the aromatic group of (xii); and
d. R23 is a saturated or unsaturated straight chain or branched optionally substituted aliphatic carbon chain.

### BRIEF DESCRIPTION OF THE DRAWINGS

FIG. 1A shows representative platelet aggregation traces and responses to increasing concentrations of CRV431. Aggregation was induced by collagen (1 μg/ml). FIG. 1B shows summary platelet aggregometry data from N=10 independent experiments. P>0.05.

FIG. 2A shows representative platelet aggregation traces and responses to increasing concentrations of CRV431. Aggregation was induced by thrombin (0.1 U/ml) FIG. 2B shows summary platelet aggregometry data from N=9 independent experiments. P>0.05.

FIGS. 3A-B show platelet aggregation traces for a single blood donor demonstrating that although CRV431 at the highest concentration tested does not inhibit platelet aggregation (FIG. 3A), platelet aggregation is inhibited by acetylsalicylic acid (ASA) (FIG. 3B).

FIG. 4A shows representative flow cytometry histograms demonstrating increasing concentrations of CRV431 reduce the percentage of phosphatidylserine (PS)-positive platelets induced by collagen (10 μg/ml) and thrombin (0.1 U/ml). FIG. 4B shows summary flow cytometry data from N=8 independent experiments. **, P<0.01 vs. vehicle control; ***, P<0.001 vs. vehicle control.

FIGS. 5A-B show data for platelet PS exposure in the presence of collagen 1 μg/ml and thrombin 0.1 U/ml (FIG. 5A) and collagen 10 μg/ml and thrombin 0.1 U/ml (FIG. 5B).

Throughout the drawings, reference numbers may be re-used to indicate correspondence between referenced elements. The drawings are provided to illustrate example embodiments described herein and are not intended to limit the scope of the disclosure.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

### Definitions

Unless defined otherwise, technical and scientific terms used herein have the same meaning as commonly understood by one of ordinary skill in the art to which the present disclosure belongs. See, e.g. Singleton et al., Dictionary of Microbiology and Molecular Biology 2nd ed., J. Wiley & Sons (New York, NY 1994); Sambrook et al., Molecular Cloning, A Laboratory Manual, Cold Spring Harbor Press (Cold Spring Harbor, NY 1989). For purposes of the present disclosure, the following terms are defined below.

As used herein, a “subject” refers to an animal that is the object of treatment, observation or experiment. “Animals” include cold- and warm-blooded vertebrates and invertebrates such as fish, shellfish, reptiles and, in particular, mammals. “Mammal” includes, without limitation, mice; rats; rabbits; guinea pigs, dogs; cats, sheep; goats, cows; horses; primates, such as monkeys, chimpanzees, and apes, and, in particular, humans.

As used herein, a “patient” refers to a subject that is being treated by a medical professional, such as a Medical Doctor (i.e., Doctor of Allopathic medicine or Doctor of Osteopathic medicine) or a Doctor of Veterinary Medicine, to attempt to cure, or at least ameliorate the effects of, a particular disease or disorder or to prevent the disease or disorder from occurring in the first place.

As used herein, “administration” or “administering” refers to a method of giving a dosage of a pharmaceutically active ingredient to a vertebrate.

As used herein, a “dosage” refers to the combined amount of the active ingredients (e.g., cyclosporine analogues, including CRV431).

As used herein, a “unit dosage” refers to an amount of therapeutic agent administered to a patient in a single dose.

As used herein, a “daily dosage” refers to the total amount of therapeutic agent administered to a patient in a day.

As used herein, “therapeutically effective amount” or “pharmaceutically effective amount” is meant an amount of therapeutic agent, which has a therapeutic effect. The dosages of a pharmaceutically active ingredient which are useful in treatment when administered alone or in combination with one or more additional therapeutic agents are therapeutically effective amounts. Thus, as used herein, a therapeutically effective amount means an amount of therapeutic agent which produces the desired therapeutic effect as judged by clinical trial results and/or model animal studies.

As used herein, the term “treat,” “treatment,” or “treating,” refers to administering a therapeutic agent or pharmaceutical composition to a subject for prophylactic and/or therapeutic purposes. The term “prophylactic treatment” refers to treating a subject who does not yet exhibit symptoms of a disease or condition, but who is susceptible to, or otherwise at risk of, a particular disease or condition, whereby the treatment reduces the likelihood that the patient will develop the disease or condition. The term “therapeutic treatment” refers to administering treatment to a subject already suffering from a disease or condition. As used herein, a “therapeutic effect” relieves, to some extent, one or more of the symptoms of a disease or disorder. For example, a therapeutic effect may be observed by a reduction of the subjective discomfort that is communicated by a subject (e.g., reduced discomfort noted in self-administered patient questionnaire).

As used herein, the term “prophylaxis” or “prevention” refers the preventive treatment of a subclinical disease-state in a subject, e.g., a mammal (including a human), for reducing the probability of the occurrence of a clinical disease-state. The subject is selected for preventative therapy based on factors that are known to increase risk of suffering a clinical disease state compared to the general population. “Prophylaxis” therapies can be divided into (a) primary prevention and (b) secondary prevention. Primary prevention is defined as treatment in a subject that has not yet presented with a clinical disease state, whereas secondary prevention is defined as preventing a second occurrence of the same or similar clinical disease state.

As used herein, the term “thrombosis” refers to formation or presence of a thrombus or thrombi; clotting within a blood vessel that may cause ischemia or infarction of tissues supplied by the vessel. The term “embolism” refers to sudden blocking of an artery by a clot or foreign material that has been brought to its site of lodgment by the blood current. The term “thromboembolism” refers to obstruction of a blood vessel with thrombotic material carried by the blood stream from the site of origin to plug another vessel. The term “thromboembolic disorders” encompasses both “thrombotic” and “embolic” disorders.

As used herein, the terms “thromboembolic disorder(s)” and “thrombotic disorder(s)” are used interchangeably, which include, but are not limited to, arterial cardiovascular thromboembolic disorders, venous cardiovascular or cerebrovascular thromboembolic disorders, and thromboembolic disorders in the chambers of the heart or in the peripheral circulation. The terms “thromboembolic disorders” and “thrombotic disorder(s)” also include specific disorders selected from, but not limited to, unstable angina or other acute coronary syndromes, atrial fibrillation, first or recurrent myocardial infarction, ischemic sudden death, transient ischemic attack, stroke, atherosclerosis, peripheral occlusive arterial disease, venous thrombosis, deep vein thrombosis, thrombophlebitis, arterial embolism, coronary arterial thrombosis, cerebral arterial thrombosis, cerebral embolism, kidney embolism, pulmonary embolism, and thrombosis resulting from medical implants, devices, or procedures in which blood is exposed to an artificial surface that promotes thrombosis. The medical implants or devices include, but are not limited to: prosthetic valves, artificial valves, indwelling catheters, stents, blood oxygenators, shunts, vascular access ports, ventricular assist devices and artificial hearts or heart chambers, and vessel grafts. The procedures include, but are not limited to: cardiopulmonary bypass, percutaneous coronary intervention, and hemodialysis. In some embodiments, the term “thromboembolic disorders” includes acute coronary syndrome, stroke, deep vein thrombosis, and pulmonary embolism.

As used herein, the term “stroke” refers to embolic stroke or atherothrombotic stroke arising from occlusive thrombosis in the carotis communis, carotid intima, or intracerebral arteries.

### Thrombosis

Hemostasis is a physiological response to vascular injury to control blood loss. Vascular injury initiates rapid activation and aggregation of platelets in the process of primary hemostasis, resulting in an unstable platelet plug. Secondary hemostasis involves activation of the plasma borne coagulation factors and is required to form a fibrin meshwork to stabilize the platelet plug. When this process occurs in excess, or at the wrong site, thrombosis occurs. Thrombosis can occur in the arterial or venous systems, leading to clinical outcomes including myocardial infarction, stroke, deep venous thrombosis. In thrombosis, a blood clot, or thrombus, may form and obstruct circulation locally, causing ischemia and organ damage. Alternatively, in a process known as embolism, the clot may dislodge and subsequently become trapped in a distal vessel, where it again causes ischemia and organ damage. Diseases arising from pathological thrombus formation are collectively referred to as thromboembolic disorders (or thrombotic disorders), which include, but are not limited to, acute coronary syndrome, unstable angina, myocardial infarction, thrombosis in the cavity of the heart, ischemic stroke, deep vein thrombosis, peripheral occlusive arterial disease, transient ischemic attack, and pulmonary embolism. Thrombosis can also occur on artificial surfaces in contact with blood, including catheters, stents, artificial heart valves, and hemodialysis membranes. Exemplary conditions contributing to the risk of developing thrombosis include alterations of the vessel wall, changes in the flow of blood, and alterations in the composition of the vascular compartment.

Activated platelets have a dual role in hemostasis and thrombosis. They aggregate to form the platelet plug and also provide the surface for the assembly of the coagulation factors. A subset of activated platelets, called “procoagulant platelets.” have distinct properties including the ability to support thrombin generation. Patients with a higher level of procoagulant platelets following a large-artery stroke were found to have a higher risk of stroke recurrence. In patients with sepsis, platelet mitochondrial membrane depolarization, which is a pre-requisite for formation of procoagulant subset of platelets, correlated with disease severity and disease outcome. The antithrombotic agents disclosed herein can, in some embodiments, prevent formation of proagulant platelets but do not inhibit platelet aggregation. In some embodiments, the antithrombotic agent is administered to a subject in need for treating arterial or venous thrombosis, including those associated with coronary artery disease, stroke, venous thromboembolism, autoimmune vasculitis, and sepsis.

Various assays are currently used in determining of efficacy of anti-thrombotic agents, including but are not limited to, in vitro enzymatic assays, in vitro clotting and coagulation assays, and in vitro models of thrombus. Assays for determining the effectiveness of anti-coagulant therapies include activity assays for the components of the coagulation signaling pathway (e.g., Factor XIIa and thrombin), and clotting assays. Assays for determining effectiveness of anti-platelet therapies include several platelet activation and adhesion assays. Flow cytometry is also often used to assess platelet activation. The in vivo assays can be common for testing all drug classes, as the common goal is to prevent and/or treat thrombus formation. There are several models for these which are also included below. These risk factors are collectively known as Virchow's triad.

The antithrombotic effects of the antithrombotic agents disclosed herein can be determined and measured using any known methods (including in vitro, ex vivo and in via methods/assays) for determining and/or measuring antithrombotic activities. For example, the effects of an antithrombotic agent as an inhibitor of the coagulation Factors XIa, VIIa, IXa, Xa, XIIa, plasma kallikrein or thrombin, can be determined using methods known in the art, for example in vitro methods using a relevant purified serine protease, respectively, and an appropriate synthetic substrate. The rate of hydrolysis of the chromogenic or fluorogenic substrate by the relevant serine protease can be measured in the absence and presence of an antithrombotic agent. In some embodiments, hydrolysis of the substrate resulted in the release of pNA (para nitroaniline), which is monitored spectrophotometrically by measuring the increase in absorbance at 405 nm, or the release of AMC (amino methylcoumarin), which was monitored spectrofluorometrically by measuring the increase in emission at 460 nm with excitation at 380 nm. A decrease in the rate of absorbance or fluorescence change in the presence of inhibitor is indicative of enzyme inhibition. The results of this assay are expressed as the inhibitory constant, Ki. An antithrombotic agent disclosed herein can be the inhibitor for one or more coagulation Factors XIa, VIIa, IXa, Xa, XIIa, plasma kallikrein, and thrombin. In some embodiments, the antithrombotic agent is the inhibitor for all of coagulation Factors XIa, VIIa, IXa, Xa, XIIa, plasma kallikrein, and thrombin. In some embodiments, the antithrombotic agent is not the inhibitor for at least one of coagulation Factors XIa, VIIa, IXa, Xa, XIIa, plasma kallikrein, and thrombin. In some embodiments, the antithrombotic agent is not the inhibitor for at least two or more of coagulation Factors XIa, VIIa, IXa, Xa, XIIa, plasma kallikrein, and thrombin.

In some embodiments, the effect of an antithrombotic agent disclosed herein as an inhibitor of coagulation is determined using a standard or modified clotting assay. For example, an increase in the plasma clotting time in the presence of inhibitor is indicative of anticoagulation. Relative clotting time is the clotting time in the presence of an inhibitor divided by the clotting time in the absence of an inhibitor. The results of this assay may be expressed as IC1.5× or IC2×, the inhibitor concentration required to increase the clotting time by 1.5-time or 2-times, respectively, relative to the clotting time in the absence of the inhibitor. The IC1.5× or IC2× is found by linear interpolation from relative clotting time versus inhibitor concentration plots using inhibitor concentration that spans the IC1.5× or IC2×. Clotting times are determined using citrated normal human plasma as well as plasma obtained from an animal such as rat or rabbit. Plasma clotting assays can be performed in an automated coagulation analyzer (Sysmex, Dade-Behring, Ill.). Similarly, clotting times can be determined from the animal or humans dosed with an antithrombotic agent.

The antithrombotic agents disclosed herein can be tested in vitro, ex vivo and in vivo for their ability to inhibit platelet aggregation, including platelet aggregation induced by gamma-thrombin. For example, platelet aggregation can be monitored in a 96-well microplate aggregation assay format or using standard platelet aggregometer. In some embodiments, the antithrombotic agent is tested using an alpha-thrombin-induced platelet aggregation assay or a tissue factor-induced platelet aggregation assay. In some embodiments, the antithrombotic agent inhibits platelet aggregation. In some embodiments, the antithrombotic agent does not inhibit platelet aggregation. In some embodiments, the antithrombotic agent reduces platelet aggregation.

The antithrombotic effect of an antithrombotic agent disclosed herein can be determined using relevant in vivo thrombosis models, including but not limited to, in vivo electrically-induced carotid artery thrombosis models, FeCl₃-induced carotid artery thrombosis, arteriovenous-shunt thrombosis models. For example, the rabbit ECAT model described by Wong et al. (J. Pharmacol Exp. Ther., 295:212-218 (2000)) and the rabbit AV shunt model described by Wong et al. (Wong, P. C. et al., J. Pharmacol Exp. Ther 292:351-357 (2000)) can be used.

### Methods of Prevention and Treatment

Disclosed herein include methods of treating a thromboembolic disorder, and methods of preventing (including primary prophylaxis and secondary prophylaxis) a thromboembolic disorder. The methods of the present disclosure can be used to reduce or prevent thrombi formation in some embodiments. The methods can be used to reduce or inhibit procoagulant platelet formation.

In some embodiments, the method comprises: administering to a subject in need thereof a composition (e.g., a SMEDD) comprising a cyclosporine analogue (e.g., CRV431), or a pharmaceutically acceptable salt, solvate, or stereoisomer thereof, wherein the subject in need thereof is a subject that is at a risk of suffering from a thromboembolic disorder, or a subject that is suffering from a thromboembolic disorder. In some embodiments, thrombi formation is prevented from occurring. In some embodiments, thrombi formation is reduced. For example, thrombi formation is reduced by at least, or at least about, 5%, 5%, 10%, 15%, 20%, 25%, 30%, 35%, 40%, 45%, 50%, 55%, 60%, 65%, 70%, 75%, 80%, 85%, 90%, 95%, or more. In some embodiments, the likelihood of thrombi formation is delayed or reduced. For example, the likelihood of thrombi formation is reduced or delayed by at least, or at least about, 5%, 5%, 10%, 15%, 20%, 25%, 30%, 35%, 40%, 45%, 50%, 55%, 60%, 65%, 70%, 75%, 80%, 85%, 90%, 95%, or more in some embodiments, the onset of thrombi formation is delayed. The delay can be, for example, seconds, minutes, hours, days, weeks or months. In some embodiments, the onset of thrombi formation is delayed by at least, or at least about, one, two, three, four, five, six, seven, eight, nine, ten, or more minutes in some embodiments, the onset of thrombi formation is delayed by at least, or at least about, one, two, three, four, five, six, seven, eight, nine, ten, or more hours.

Antithrombotic agents disclosed herein can be used for primary and secondary prevention (i.e., prophylaxis or risk reduction) of thromboembolic disorders, as well as treatment of an already existing thrombotic process. For example, the antithrombotic agents can be administered to patients at risk of developing thromboembolic disease because of the presence of one or more predisposing risk factors from Virchow's triad to prevent formation of an occlusive thrombus (primary prevention). For example, in an orthopedic surgery setting (e.g., hip and knee replacement), the antithrombotic agent can be administered prior to a surgical procedure. The antithrombotic agent can counterbalance the prothrombotic stimulus exerted by vascular flow alterations (stasis), potential surgical vessel wall injury, as well as changes in the composition of the blood due to the acute phase response related to surgery. The antithrombotic agent can also be used in patients at risk for developing thrombotic cardiovascular disease.

Antithrombotic agents disclosed herein can also be used in secondary prevention, following an initial thrombotic episode. For example, patients with mutations in factor V (also known as factor V Leiden) and additional risk factors (e.g., pregnancy), can be administered with the antithrombotic agent to prevent the reoccurrence of venous thrombosis. In some embodiments, the antithrombotic agent is used in secondary prevention of cardiovascular events in patients with a history of acute myocardial infarction or acute coronary syndrome.

The antithrombotic agents disclosed herein, in some embodiments, are administered to a subject in need to treat the disease state (i.e., by arresting its development) after it has already started. For example, patients presenting with deep vein thrombosis are treated with the antithrombotic agent to prevent further growth of the venous occlusion. In some embodiments, over time, the antithrombotic agent can cause a regression of the disease state because the balance between prothrombotic factors and anticoagulant/profibrinolytic pathways is changed in favor of the latter. Examples on the arterial vascular bed include the treatment of patients with acute myocardial infarction or acute coronary syndrome with aspirin and clopidogrel to prevent further growth of vascular occlusions and eventually leading to a regression of thrombotic occlusions.

The antithrombotic agents disclosed herein, in some embodiments, are administered to a subject in need to prevent or to reduce “contact activation” of coagulation which happens when blood is exposed to artificial surfaces (e.g., during hemodialysis, “on-pump” cardiovascular surgery, vessel grafts, bacterial sepsis), on cell surfaces, cellular receptors, cell debris, DNA, RNA, and extracellular matrices.

Thrombosis can include, but is not limited to, vessel occlusion (e.g., after a bypass) and reocclusion (e.g., during or after percutaneous transluminal coronary angioplasty). The thromboembolic disorders can result from conditions, for example, atherosclerosis, surgery or surgical complications, prolonged immobilization, arterial fibrillation, congenital thrombophilia, cancer, diabetes, effects of medications or hormones, and complications of pregnancy. In some embodiments, the thromboembolic disorder is associated with patients with atherosclerosis. In some embodiments, arterial fibrillation and subsequent thromboembolic disorders include cardiovascular disease, rheumatic heart disease, nonrheumatic mitral valve disease, hypertensive cardiovascular disease, chronic lung disease, cardiac abnormalities, and thyrotoxicosis. In some embodiments, the thromboembolic disorder is associated with diabetes mellitus. In some embodiments, the thromboembolic disorder is associated with congenital thrombophilia which can be caused by or associated with gain of function mutations in coagulation factors or loss of function mutations in the anticoagulant- or fibrinolytic pathways.

Thrombosis can be associated with one or more tumor type, e.g., pancreatic cancer, breast cancer, brain tumors, lung cancer, ovarian cancer, prostate cancer, gastrointestinal malignancies, and Hodgkins or non-Hodgkins lymphoma. In some embodiments, thrombosis is associated with prostate cancer, colorectal cancer, brain cancer, lung cancer, breast cancer, ovarian cancer, or a combination thereof. In some embodiments, the thromboembolic disorder is venous thromboembolism (VTE) in cancer patients. The risk under which a cancer patient is for thrombosis can vary because of one or more of the following: (i) the stage of the cancer (i.e., presence of metastases), (ii) the presence of central vein catheters, (iii) surgery and anticancer therapies including chemotherapy, and (iv) hormones and antiangiogenic drugs. In some embodiments, one or more of the antithrombotic agents disclosed is administered to a patient having advanced tumors to prevent thromboembolic disorders. In some embodiments, the antithrombotic agent is administered to a cancer patient bedridden for prolonged periods of time, an ambulatory patient who is receiving chemotherapy or radiation, a cancer patient with indwelling central vein catheters, or a cancer patient undergoing surgery.

As described herein, cyclosporine analogues (for example CRV431) can be used to treat a thromboembolic disorder or to prevent (e.g., as primary prophylaxis or secondary prophylaxis) of a thromboembolic disorder. The cyclosporine analogues can prevent the thromboembolic disorder (or reduce or prevent thrombi formation, or reduce or inhibit procoagulant platelet formation), in some embodiments, without causing bleeding risk or increasing the bleeding risk, in a subject in need thereof. The cyclosporine analogues can also be used for reducing or preventing thrombi formation. The cyclosporine analogues can also be used for reducing or inhibiting procoagulant platelet formation. In some embodiments, the cyclosporine analogues can prevent or treat the thromboembolic disorder without inhibiting platelet aggregation. In some embodiments, the cyclosporine analogues can prevent or treat the thromboembolic disorder without completely inhibiting platelet aggregation (e.g., platelet aggregation is reduced by about or by at most about 85%, 80%, 70%, 60%, 50%, 45%, 40%, 35%, 30%, 25%, 20%, 15%, 10%, 5%, or less in the subject as compared to an untreated subject). In some embodiments, the cyclosporine analogues can reduce the bleeding risk (e.g., by or by at least 5%, 10%, 15%, 20%, 25%, 30%, 35%, 40%, 45%, 50%, 55%, 60%, 65%, 70%, 75%, 80%, 85%, 0.0%, 95%, or more as compared to an untreated subject). In some embodiments, the cyclosporine analogues can reduce thrombi formation, delay or reduce the likelihood of thrombi formation, prevent or delay the onset of thrombi formation, and/or reduce the bleeding risk without inhibiting procoagulant platelet formation (or with selective inhibition of procoagulant platelet formation) and/or without inhibiting platelet aggregation (or with reduced inhibition of platelet aggregation or with partial inhibition of platelet agreement, such as reducing platelet aggregation by, or by at most 85%, 80%, 70%, 60%, 50%, 45%, 40%, 35%, 30%, 25%, 20%, 15%, 10%, 5%, or less in the subject as compared to an untreated subject).

### Disorder

In some embodiments, a cyclosporine analogue is used to treat or prevent a thromboembolic disorder in a subject. For example, the thromboembolic disorder can be an arterial cardiovascular thromboembolic disorder, a venous cardiovascular thromboembolic disorder, or a thromboembolic disorder in a chamber of the heart or in the peripheral circulation. The thromboembolic disorder can be, for example, unstable angina, an acute coronary syndrome, myocardial infarction, transient ischemic attack, stroke, atherosclerosis, peripheral occlusive arterial disease, venous thrombosis, deep vein thrombosis, thrombophlebitis, arterial embolism, coronary arterial thrombosis, cerebral arterial thrombosis, cerebral embolism, kidney embolism, pulmonary embolism, or thrombosis resulting from an medical implant, device, or procedure in which blood is exposed to an artificial surface that promotes thrombosis. As another example, the thromboembolic disorder is stroke, myocardial infarction, unstable angina, abrupt closure following angioplasty or stent placement, thrombosis induced by a peripheral vascular surgery or a peripheral vascular disease, or a thrombotic disorder resulting from atrial fibrillation or inflammation.

In some embodiments, a cyclosporine analogue is used to reduce or inhibit procoagulant platelet formation in a subject. The subject suffers from or is at risk of developing a prothrombotic or thrombotic condition. The prothrombotic or thrombotic condition can be selected from the group consisting of infection, sepsis, systemic inflammatory response syndrome, multi organ failure, thrombotic thrombocytopenia purpura, hemolytic uremia syndrome, vascularization, renal failure, ischemic repercussion injury, solid organ transplant rejection, cardiovascular disease, stroke, venous thromboembolism, autoimmune disorders, sickle cell disease, inflammatory bowel disease, acute lung injury, malignancy, myocardial infarction (primary and secondary), embolic stroke, ischemic stroke, thrombotic stroke, deep vein thrombosis (DVT), thromboembolism, portal vein thrombosis, renal vein thrombosis, jugular vein thrombosis, Budd-Chiari syndrome, Paget-Schroetter disease, cerebral venous sinus thrombosis, arterial thrombosis, and arterial embolism.

### Therapeutic Agents

Cyclosporine is also known as cyclosporine A (CsA). In some embodiments, a cyclosporine analogue is a compound of Formula L:

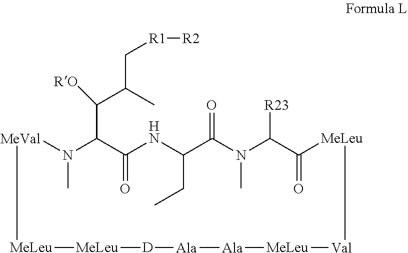

wherein
a. R is H or acetyl;
b. R1 is a saturated or unsaturated straight or branched aliphatic carbon chain from 2 to 15 carbon atoms in length;
c. R2 is selected from the group consisting of:
   i. H;
   ii. an unsubstituted, N-substituted, or N,N-disubstituted amide;
   iii. a N-substituted or unsubstituted acyl protected amine;
   iv. a N-substituted or unsubstituted amine;
   v. a carboxylic acid;
   vi. a nitrile;
   vii. an ester;
   viii. a ketone;
   ix. a hydroxy, dihydroxy, trihydroxy, or polyhydroxy alkyl; and
   x. a substituted or unsubstituted aryl;
   xi. a saturated or unsaturated, straight or branched aliphatic chain optionally containing a substituent selected from the group consisting of a hydrogen, a ketone, a hydroxyl, a nitrile, a carboxylic acid, an ester, a 1,3-dioxolane, a halogen, and an oxo;
   xii. an aromatic group containing a substituent selected from the group consisting of a halogen, an ester, and a nitro; and
   xiii. a combination of the saturated or unsaturated, straight or branched aliphatic chain of (xi) and the aromatic group of (xii); and
d. R23 is a saturated or unsaturated straight chain or branched optionally substituted aliphatic carbon chain.

In some embodiments, R1-R2 is selected from the group consisting of:

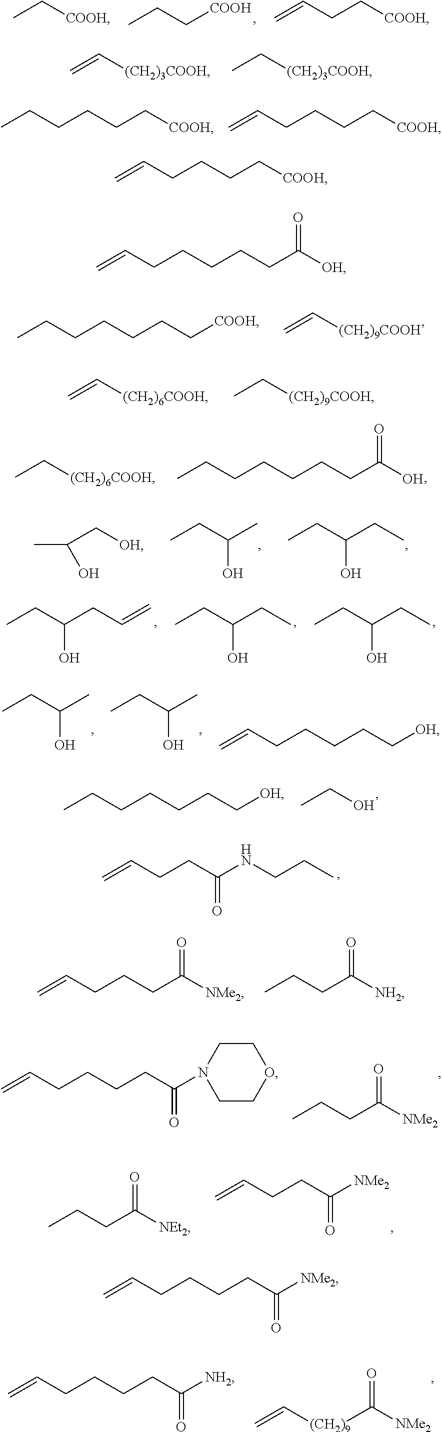

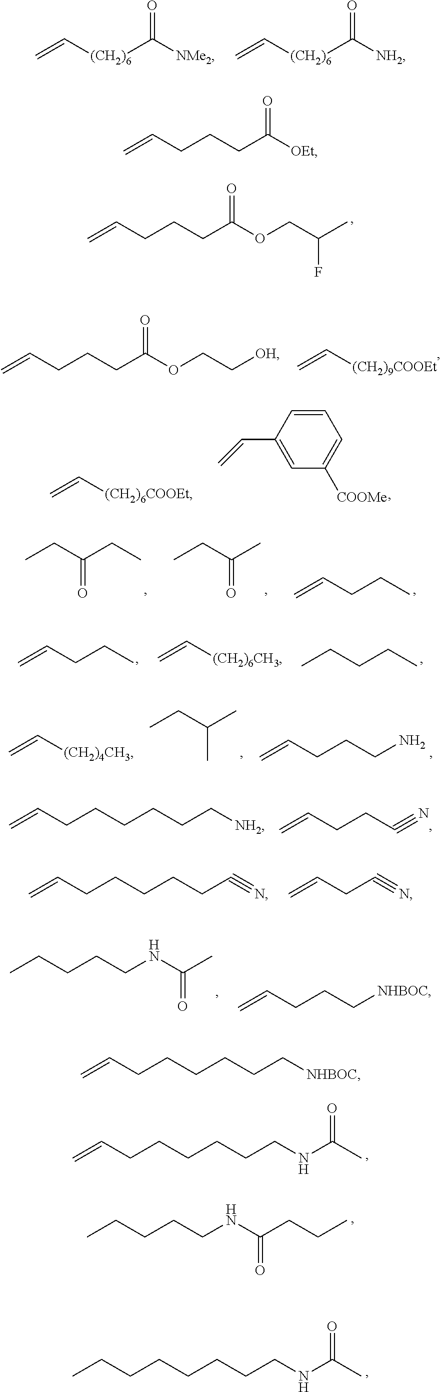

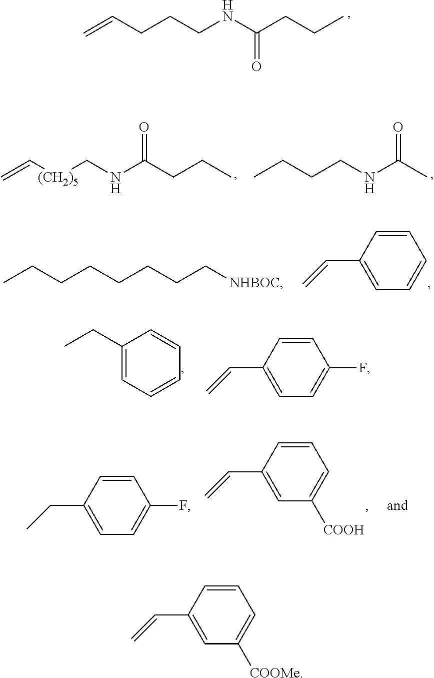

In some embodiments, R1-R2 comprises a saturated or unsaturated, straight or branched aliphatic chain of between 2 and 5 carbons optionally substituted with a substituent selected from the group consisting of a hydrogen, a ketone, a hydroxyl, a nitrile, a halogen, an oxo, a carboxylic acid, an ester, and an 1,3-dioxolane.

In some embodiments, R2 is selected from the group consisting of

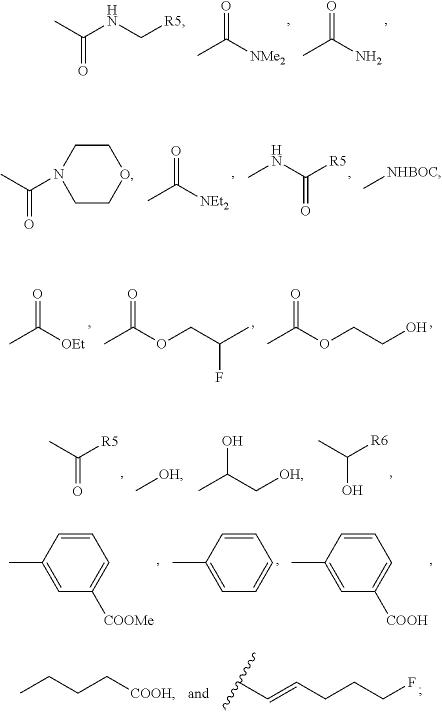

R5 is a saturated or unsaturated straight or branched aliphatic carbon chain between 1 and 10 carbons in length; and R6 is a monohydroxylated, dihydroxylated, trihydroxylated or polyhydroxylated saturated or unsaturated straight chain or branched aliphatic carbon chain between 1 and 10 carbons in length.

In some embodiments, R23 is selected from the group consisting of —CH₃—CH₂CH₃—CH₂CHCH₂—CH₂CH₂CH₂I, CH₂)₃CH₂I, —(CH₂)₃N⁺(CH₃)₃, —CH₂CCH, —CH₂CO₂(t-Bu), —CH₂Ph, —CH₂OH, —CH(OH)CH₃, —CH(OH)(t-Bu), —CH(OH)Ph, —COOH, —SCH₃, and —S(p-Tol). In some embodiments, R23 comprises an optionally substituted alkyl, including optionally substituted C1-C3 alkyl. The alkyl can be substituted with amino and may comprise a C1-C3-Ala, wherein the compound comprises the D-epimer of amino acid 3 which is the amino acid to which R23 is attached. In some embodiments, R23 can be MeAla. In some embodiments, R23 is a straight or branched aliphatic carbon chain of 1 to 6, 1 to 5, 1 to 4, 1 to 3 or 2 carbons in length.

In some embodiments,

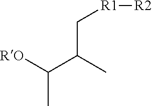

in Formula L is selected from the group consisting of:

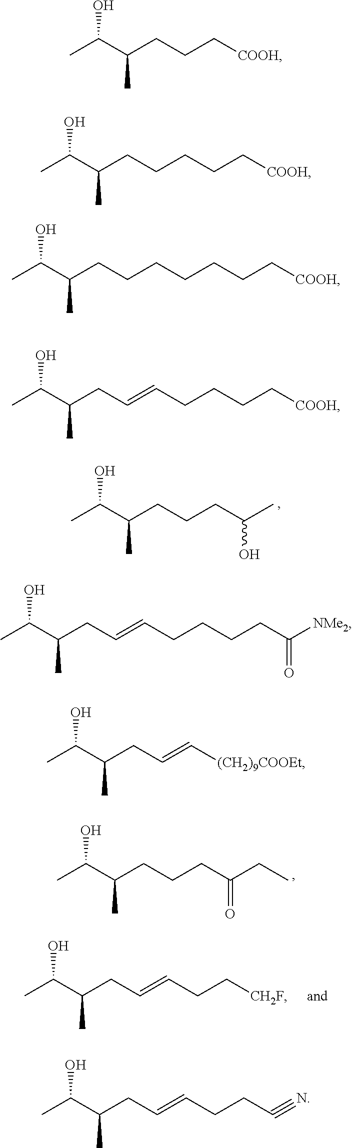

In some embodiments, a cyclosporine analogue is a compound of Formula L:

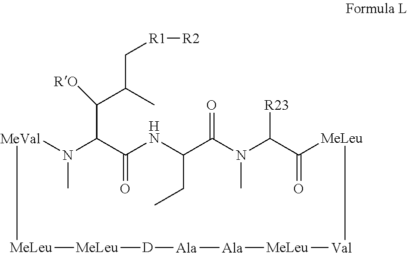

wherein:
a. R′ is H or acetyl;
b. R1 is a saturated or unsaturated straight or branched aliphatic carbon chain from 2 to 15 carbon atoms in length;
c. R2 is selected from the group consisting of:
   i. an unsubstituted, N-substituted, or N,N-disubstituted amide;
   ii. a carboxylic acid;
   iii. a nitrile;
   iv. an ester;
   v. a ketone;
   vi. a hydroxy, dihydroxy, trihydroxy, or polyhydroxy alkyl;
   vii. a substituted or unsubstituted aryl;
   viii. a saturated or unsaturated straight or branched aliphatic carbon chain substituted with a substituent selected from the group consisting of a ketone, a hydroxy, a nitrile, a carboxylic acid, an ester, a 1,3-dioxolane, and an oxo;
   ix. an aromatic group substituted with a substituent selected from the group consisting of a halogen, an ester, and a nitro; and
   x. a combination of the saturated or unsaturated straight or branched aliphatic carbon chain of viii) and the aromatic group of ix); and
d. R23 is unsubstituted C₁-C₃ alkyl.

In some embodiments, R′ is H.

In some embodiments, R1 is a saturated or unsaturated straight or branched aliphatic carbon chain from 5 to 8 carbon atoms in length.

In some embodiments, R2 is selected from the group consisting of:

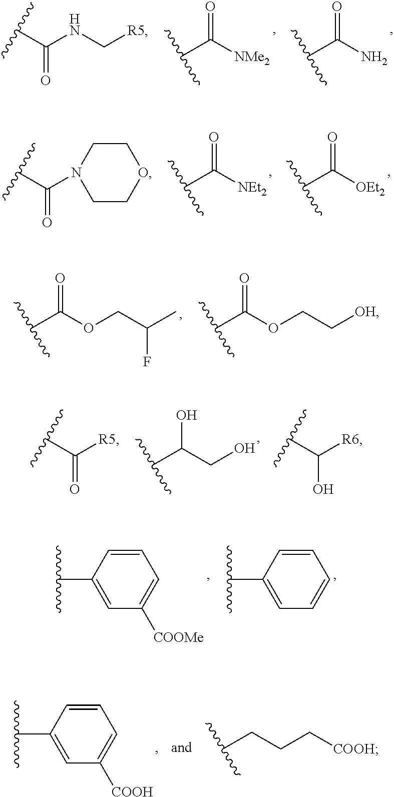

R5 is a saturated or unsaturated straight or branched aliphatic carbon chain between 1 and 10 carbons in length, and R6 is a monohydroxylated, dihydroxylated, trihydroxylated, or polyhydroxylated saturated or unsaturated straight or branched aliphatic carbon chain between 1 and 10 carbons in length.

In some embodiments, R1-R2 is selected from the group consisting of:

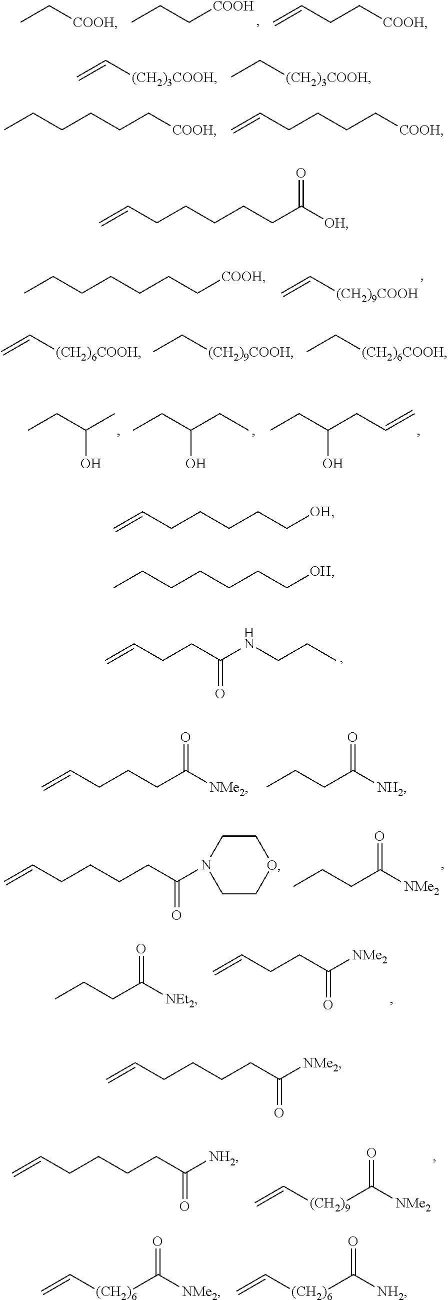

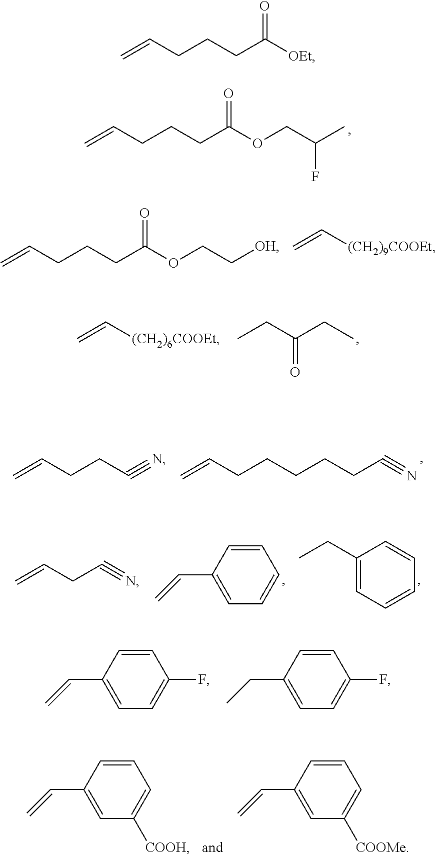

In some embodiments, R1-R2 is substituted with a substituent selected from the group consisting of a ketone, a hydroxy, a nitrile, an oxo, a carboxylic acid, an ester, and a 1,3-dioxolane. In some embodiments, R1-R2 is at least 6 carbon atoms in length.

In some embodiments,

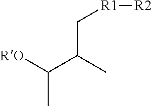

in Formula L is selected from the group consisting of:

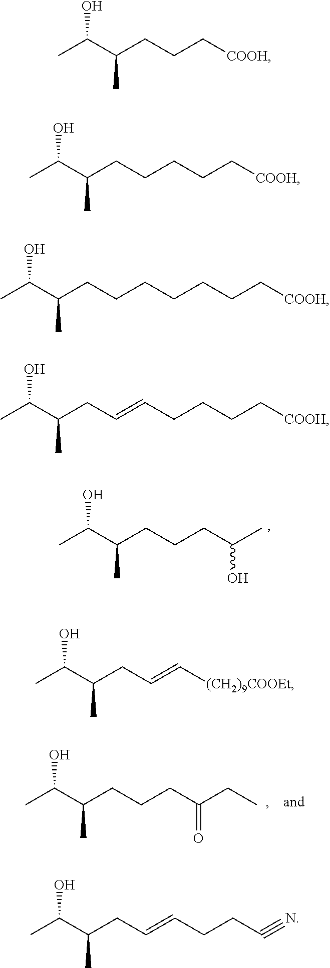

In some embodiments, R23 is selected from the group consisting of: —CH₃ and —CH₂CH₃. In some embodiments, R23 is methyl. In some embodiments, the compound comprises the D-epimer of amino acid 3 which is the amino acid to which R23 is attached.

In some embodiments, a cyclosporine analogue is a compound selected from the group consisting of:

| | | R23 | Isomer |
|---|---|---|---|
| a) | 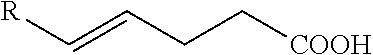 | —CH₃ | L |
| b) | 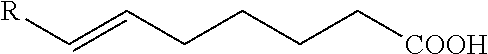 | —CH₃ | D |
| c) | 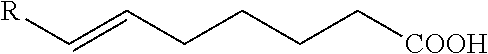 | —CH₃ | L |
| d) | 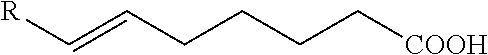 | —CH₂CH₃ | D |
| e) | 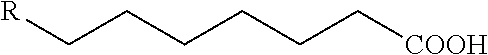 | —CH₃ | D |
| f) | 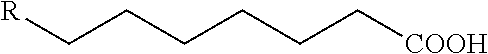 | —CH₃ | L |
| g) | 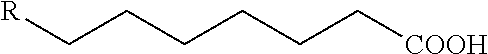 | —CH₂CH₃ | D |
| h) | 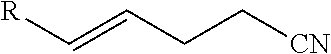 | —CH₃ | D |
| i) | 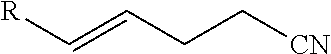 | —CH₂CH₃ | L |
| j) | 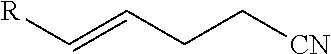 | —CH₂CH₃ | D |
| k) | 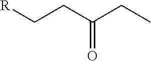 | —CH₃ | L |
| l) | 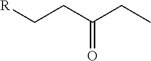 | —CH₃ | D |
| m) | 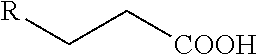 | —CH₃ | D |
| n) | 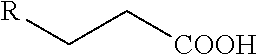 | —CH₂CH₃ | D |
| o) | 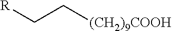 | —CH₃ | D |
| r) | 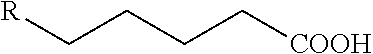 | —CH₃ | D |
| s) | 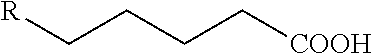 | —CH₃ | L |
| t) | 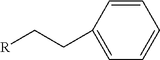 | —CH₃ | D |
| u) | 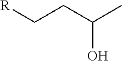 | —CH₃ | D |
| v) | 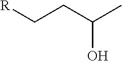 | —CH₃ | L |
| w) | 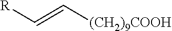 | —CH₂CH₃ | L |
| x) | 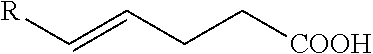 | —SCH₃ | D/L |
| z) | 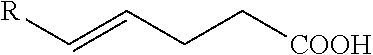 | —(CH₂)₃N⁺(CH₃)₃ | D/L |
| aa) | 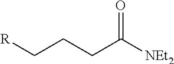 | 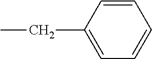 | D |
| bb) | 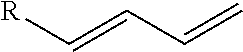 | —CH₃ | D |
| cc) | 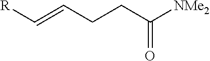 | —CH₃ | L |
| dd) | 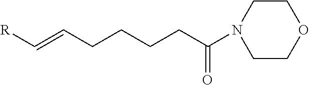 | —CH₃ | L |
| ee) | 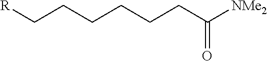 | —CH₃ | D |
| ff) | 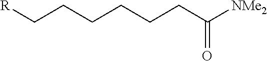 | —CH₂CH₃ | D |
| gg) | 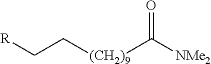 | —CH₃ | D |
| hh) | 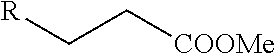 | —CH₃ | D |
| ii) | 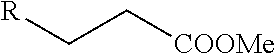 | —CH₂CH₃ | D |
| jj) | 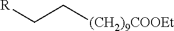 | —CH₃ | D |

wherein:
R is

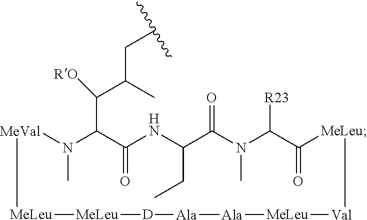

R′ is H or acetyl; and
the isomer is the isomeric form of amino acid 3 which is the amino acid to which R23 is attached.

In some embodiments, a cyclosporine analogue is a compound of Formula L:

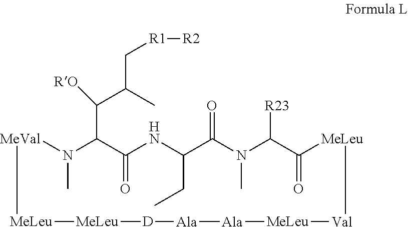

wherein:
a. R′ is H or acetyl;
b. R1 is a saturated or unsaturated straight or branched aliphatic carbon chain from 2 to 15 carbon atoms in length;
c. R2 is selected from the group consisting of.
   i. an unsubstituted, N-substituted, or N,N-disubstituted amide;
   ii. a carboxylic acid;
   iii. a nitrile;
   iv. an ester;
   v. a ketone;
   vi. a hydroxy, dihydroxy, trihydroxy, or polyhydroxy alkyl;
   vii. a substituted or unsubstituted aryl;
   viii. a saturated or unsaturated straight or branched aliphatic carbon chain substituted with a substituent selected from the group consisting of a ketone, a hydroxy, a nitrile, a carboxylic acid, an ester, a 1,3-dioxolane, and an oxo;
   ix. an aromatic group substituted with a substituent selected from the group consisting of a halogen, an ester, and a nitro; and
   x. a combination of the saturated or unsaturated straight or branched aliphatic carbon chain of (viii) and the aromatic group of (ix); and
d. R23 is a saturated or unsaturated straight or branched optionally substituted aliphatic carbon chain,
   wherein R1-R2 is at least 6 carbon atoms in length.

In some embodiments, R′ is H.

In some embodiments, R1 is a saturated or unsaturated straight or branched aliphatic carbon chain from 5 to 8 carbon atoms in length.

In some embodiments, R1-R2 is selected from the group consisting of:

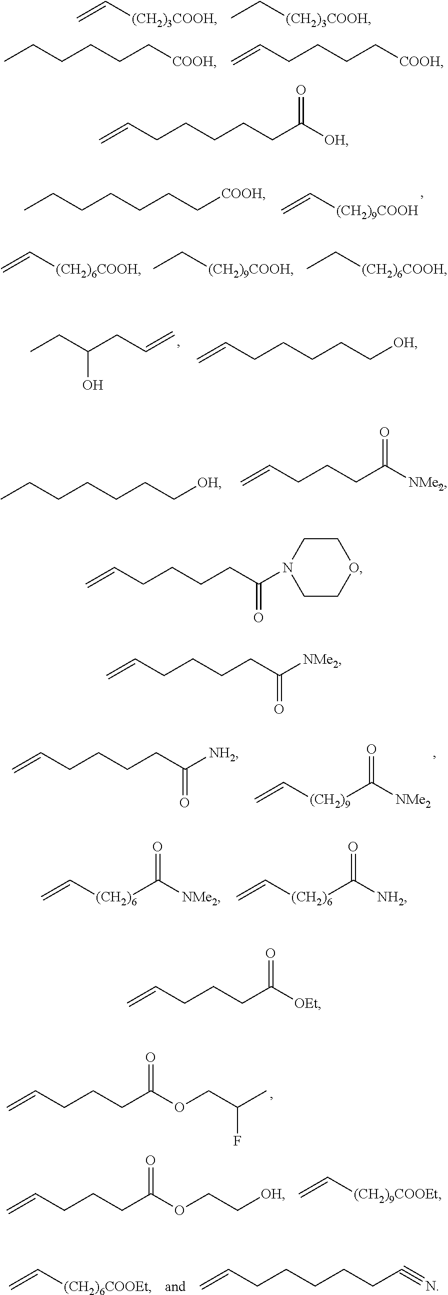

In some embodiments, R1-R2 is substituted with a substituent selected from the group consisting of a ketone, a hydroxy, a nitrile, an oxo, a carboxylic acid, an ester, and a 1,3-dioxolane.

In some embodiments, R23 is selected from the group consisting of: —CH₃, —CH₂CH₃, —CH₂CHCH₂, —CH₂CH₂CH₂I, —(CH₂)₃CH₂I, —(CH₂)₃N⁺(CH₃)₃, —CH₂CCH, —CH₂CO₂(t-Bu), —CH₂Ph, —CH₂OH, —CH(OH)CH₃, —CH(OH)(t-Bu), —CH(OH)Ph, —COOH, —SCH₃, and —S(p-Tol). In some embodiments, R23 comprises an optionally substituted C₁-C₃ alkyl. In some embodiments, R23 is substituted with amino. In some embodiments, R23 is C₁-C₃ alkyl and the compound comprises the D-epimer of amino acid 3 which is the amino acid to which R23 is attached. In some embodiments, R23 is methyl. In some embodiments, R23 is a straight or branched aliphatic carbon chain of 1 to 6 carbons in length.

In some embodiments,

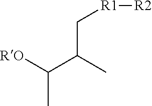

in Formula L is selected from the group consisting of:

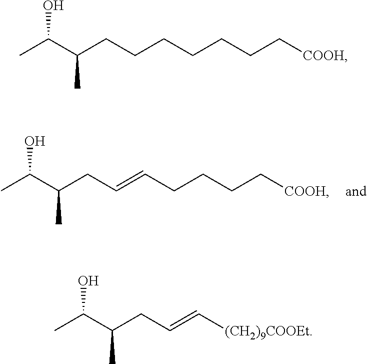

In some embodiments, R1-R2 is

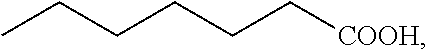

R23 is methyl, and the compound is a D-epimer of amino acid 3 which is the amino acid to which R23 is attached.

In some embodiments, a cyclosporine analogue is a compound of Formula L:

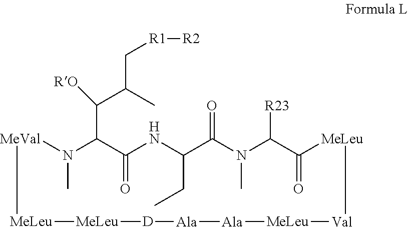

wherein:
R′ is H or acetyl;
R1 is a saturated or unsaturated straight chain or branched aliphatic carbon chain from 2 to 15 carbon atoms in length;
R2 is a N-substituted or unsubstituted acyl protected amine; and
R23 is methyl or ethyl.

In some embodiments, R′ is H.

In some embodiments, R1 is a saturated or unsaturated straight or branched aliphatic carbon chain from 5 to 8 carbon atoms in length.

In some embodiments, R2 is

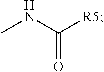

wherein R5 is a saturated or unsaturated straight or branched aliphatic carbon chain between 1 and 10 carbons in length.

In some embodiments, R1-R2 is selected from the group consisting of:

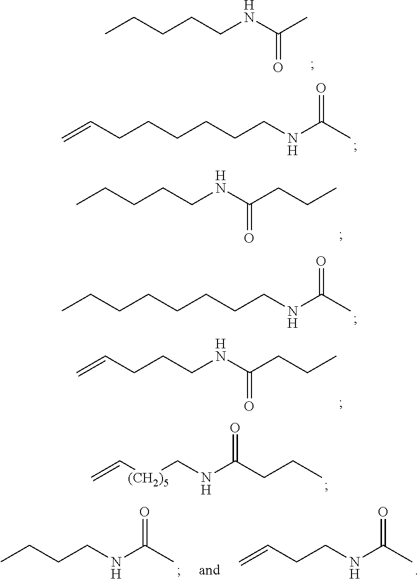

In some embodiments, R23 is methyl.

In some embodiments,

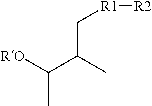

in formula L is

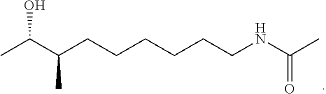

In some embodiments, R′, R1-R2, and R23 and the isomer of said compound are selected from the following:

| R′ | R1—R2 | R23 | Isomer |
|---|---|---|---|
| H | 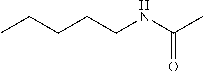 | —CH₃ | D |
| H | 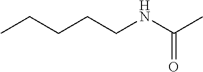 | —CH₂CH₃ | L |
| H | 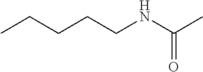 | —CH₂CH₃ | D |

wherein the isomer is the isomeric form of amino acid 3 which is the amino acid to which R23 is attached.

In some embodiments, a cyclosporine analogue is a compound of Formula L:

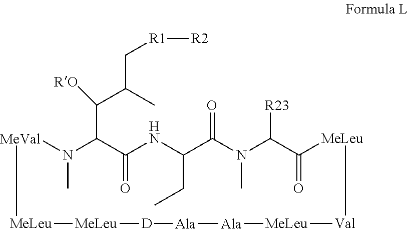

wherein:
R′ is H or acetyl;
R1-R2 is selected from the group consisting of:

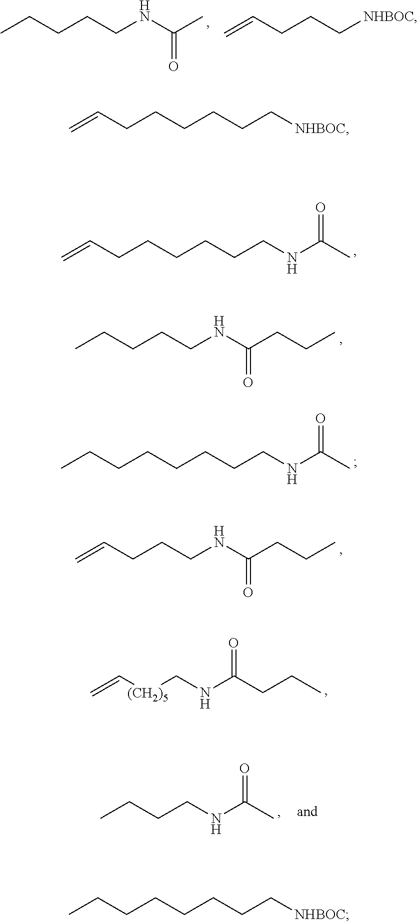

and
R23 is a saturated or unsaturated straight chain or branched optionally substituted aliphatic carbon chain.

In some embodiments, R′ is H.

In some embodiments, R1-R2 is

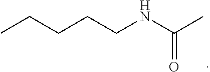

In some embodiments,

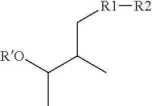

### in Formula L is

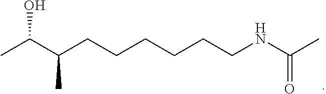

In some embodiments, R23 is selected from the group consisting of: —CH₃, —CH₂CH₃, —CH₂CHCH₂, —CH₂CH₂CH₂I, —(CH₂)₃CH₂I, —(CH₂)₃N⁺(CH₃)₃, —CH₂CCH, —CH₂CO₂(t-Bu), —CH₂Ph, —CH₂OH, —CH(OH)CH₃, —CH(OH)(t-Bu), —CH(OH)Ph, —COOH, —SCH₃, and —S(p-Tol). In some embodiments, R23 is —CH₃ or —CH₂CH₃. In some embodiments, R23 is —CH₃. In some embodiments, R23 (a) comprises an optionally substituted C1-C3 alkyl; (b) is substituted with an amino; (c) is a C1-C3-Ala and said compound comprises the D-epimer, (d) is MeAla; and/or (e) is a straight or branched aliphatic carbon chain of 1 to 6, 1 to 5, 1 to 4, 1 to 3 or 2 carbons in length.

In some embodiments, R′, R1-R2 and R23 and the isomer of said compound are selected from the following:

| R′ | R1—R2 | R23 | Isomer |
|---|---|---|---|
| H | 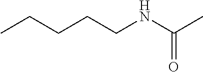 | —CH₃ | D |
| H | 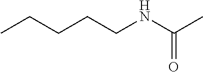 | —CH₂CH₃ | L |
| H | 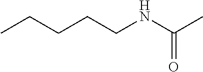 | —CH₂CH₃ | D |

In some embodiments, the cyclosporine analogue is a small molecule cyclophilin inhibitor CRV431 (shown below) which is a derivative or analogue of cyclosporine, a neutral cyclic peptide consisting of eleven amino acids, wherein amino acids 1 and 3 have been chemically modified.

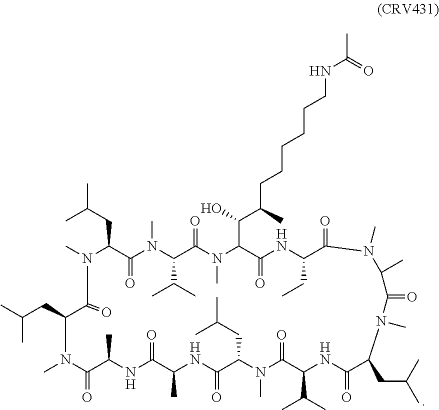 [SMILES: CC[C@@H]1NC(=O)C([C@H](O)[C@H](C)CCCCCCNC(C)=O)N(C)C(=O)[C@H](C(C)C)N(C)C(=O)[C@H](CC(C)C)N(C)C(=O)[C@H](CC(C)C)N(C)C(=O)[C@@H](C)NC(=O)[C@H](C)NC(=O)[C@H](CC(C)C)N(C)C(=O)[C@H](C(C)C)NC(=O)[C@H](CC(C)C)N(C)C(=O)C(C)N(C)C1=O]

### Additional Therapeutic Agents

In some embodiments, the method can comprise administering to the subject in need thereof one or more additional therapeutic agents. For example, the composition can comprise one or more additional therapeutic agents. Non-limiting examples of the one or more additional therapeutic agents include anti-coagulant agents, antiplatelet agents, fibrinolytic agents, or a combination thereof. In some embodiments, the one or more additional therapeutic agents comprises heparin, low molecular weight heparins, bivalirudin, Fondaparinux, warfarin, Acenocoumarol, Phenprocoumon, Phenindione, Abbokinase (urokinase), streptokinase, alteplase, retaplase, tenecteplase, prasugrel, aspirin, ticlopidine, clopidogrel, abciximab, eptifibatide, tirofiba, or a combination thereof.

In some embodiments, each of the one or more additional therapeutic agents (or all of the additional therapeutic reagents) is co-administered to the subject with the composition comprising cyclosporine analogue. In some embodiments, each of the one or more additional therapeutic agents (or all of the additional therapeutic reagents) is administered to the subject before the administration of the composition comprising cyclosporine analogue, after the administration of the composition comprising cyclosporine analogue, or both. The dosage and/or dosing schedule of an additional therapeutic agent can be the same as or different from the dosage and/or dosing schedule of the cyclosporine analogue composition described herein. The route of administration of an additional therapeutic agent can be the same as or different from the route of administration of the cyclosporine analogue composition described herein. An additional therapeutic agent can be administered by, for example, intravenous administration, oral administration, or parenteral administration.

The amount of each of the one or more additional therapeutic reagents (or all of the additional therapeutic reagents) administered can be, or be about, 0.1-1 mg (e.g., 0.1 mg, 0.5 mg or 1 mg), 1-5 mg (e.g., 1 mg, 2 mg, 3 mg, 4 mg or 5 mg), 5-10 mg (e.g., 5 mg, 6 mg, 7 mg, 8 mg, 9 mg or 10 mg), 10-20 mg (e.g., 10 mg, 15 mg or 20 mg), 20-30 mg (e.g., 20 mg, 25 mg or 30 mg), 30-40 mg (e.g., 30 mg, 35 mg or 40 mg), 40-50 mg (e.g., 40 mg, 45 mg or 50 mg), 50-100 mg (e.g., 50 mg, 60 mg, 70 mg, 80 mg, 90 mg or 100 mg), 100-150 mg (e.g., 100 mg, 125 mg or 150 mg), or 150-200 mg (e.g., 150 mg, 175 mg or 200 mg). The amount of each of the one or more additional therapeutic reagents (or all of the additional therapeutic reagents) administered can be, or be about, 1 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2 mg/kg, 2.1 mg/kg, 2.2 mg/kg, 2.3 mg/kg, 2.4 mg/kg, 2.5 mg/kg, 2.6 mg/kg, 2.7 mg/kg, 2.8 mg/kg, 2.9 mg/kg, 3 mg/kg, 3.1 mg/kg, 3.2 mg/kg, 3.3 mg/kg, 3.4 mg/kg, 3.5 mg/kg, 3.6 mg/kg, 3.7 mg/kg, 3.8 mg/kg, 3.9 mg/kg, 4 mg/kg, 4.1 mg/kg, 4.2 mg/kg, 4.3 mg/kg, 4.4 mg/kg, 4.5 mg/kg, 4.6 mg/kg, 4.7 mg/kg, 4.8 mg/kg, 4.9 mg/kg, 5 mg/kg, 6 mg/kg, 7 mg/kg, 8 mg/kg, 9 mg/kg, 10 mg/kg, 11 mg/kg, 12 mg/kg, 13 mg/kg, or a number or a range between any two of these values.

### Compositions, Kits, and Administration

Provided in some embodiments include kits comprising: a cyclosporine analogue (e.g., CRV431), such as a SMEDD of the cyclosporine analogue, or a pharmaceutically acceptable salt, solvate, stereoisomer, or composition thereof, and a label indicating the use of the kit. In some embodiments, the label indicates that the kit is for preventing a thromboembolic disorder. In some embodiments, the label indicates that the kit is for treating a thromboembolic disorder. In some embodiments, the label indicates that the kit is for reducing or inhibiting procoagulant platelet formation.

In some embodiments, the label can comprise instructions to administer the cyclosporine analogue (or a composition thereof) at a dosage, at a dosing schedule, and/or using a route of administration described herein. For example, the label comprises instructions to administer the cyclosporine analogue (or a composition thereof) to a subject by intravenous administration, oral administration, or parenteral administration. The label can comprise instructions to administer the cyclosporine analogue (or a composition thereof) to the subject by oral or intravenous administration. For example, the label can comprise instructions to administer the cyclosporine analogue (or a composition thereof) to the subject at an effective daily dose of the cyclosporine analogue or a pharmaceutically acceptable salt, solvate, or stereoisomer thereof, at from 10 mg to 250 mg.

In some embodiments, the label comprises instructions to administer the cyclosporine analogue with one or more additional therapeutic agents (e.g., an anti-coagulant agent, an antiplatelet agent, a fibrinolytic agent, or a combination thereof) described herein. In some embodiments, the kit comprises at least one of the one or more additional therapeutic agents. The label can comprise instructions to co-administer at least one of the one or more additional therapeutic agents with the cyclosporine analogue to the subject. The label can comprise instructions to administer at least one of the one or more additional therapeutic agents to the subject before the administration of the cyclosporine analogue to the subject, after the administration of the cyclosporine analogue to the subject, or both. The label can include instructions to administer the one or more additional therapeutic agents at a dosage, at dosing schedule, and/or a route of administration described herein.

Also provided herein, in some embodiments, are compositions comprising: a cyclosporine analogue (e.g., CRV431), such as a SMEDD of the cyclosporine analogue, or a pharmaceutically acceptable salt, solvate, or stereoisomer thereof for use in treating or preventing a thromboembolic disorder, for reducing or preventing thrombi formation, and/or reducing or inhibiting procoagulant platelet formation. The composition can comprise: a cyclosporine analogue (e.g., CRV431), or a pharmaceutically acceptable salt, solvate, or stereoisomer thereof for use in preventing or reducing formation of thrombi. The composition can comprise: a cyclosporine analogue (e.g., CRV431), or a pharmaceutically acceptable salt, solvate, or stereoisomer thereof, for use in reducing or inhibiting procoagulant platelet formation. The composition can be used in combination with one or more additional therapeutic agents disclosed herein. The composition can be administered at a dosage, at a dosing schedule, and/or using a route of administration described herein. The composition can be administered with one or more additional therapeutic agents disclosed herein at a dosage, at a dosing schedule, or using an administration route disclosed herein. The composition can include at least one of the one or more additional therapeutic agents. Also disclosed herein include uses of a pharmaceutically composition comprising a cyclosporine analogue for the manufacture of a medicament for treating a thromboembolic disorder, for preventing or reducing thrombi formation, and/or for reducing or inhibiting procoagulant platelet formation in a subject.

In some embodiments, the cyclosporine analogue is in or the composition is a stable self-microemulsifying drug delivery system (“SMEDDS”) formulation comprising a derivative or an analogue of cyclosporine, such as CRV431. The composition can, for example, enables good solubility of a derivative or analogue of cyclosporine (e.g., CRV431) and enables significant blood exposure in humans. In some embodiments, the composition further comprises polyoxyl castor oil (also known as polyoxyl 40 hydrogenated castor oil, macrogolglycerol hydroxystearate, and PEG-40 hydrogenated castor oil, such as Cremophor® RH40 and Kolliphor® RH40). In some embodiments, the composition further comprises ethanol. In some embodiments, the composition further comprises diethylene glycol monoethyl ether (also known as 2-(2-Ethoxyethoxy)ethanol, such as Transcutol®). In some embodiments, the composition further comprises propylene glycol (PG). In some embodiments, the composition further comprises glyceryl monolinoleate, such as Maisine® CC. In some embodiments, the composition further comprises Vitamin E. Various SMEDDS formulations of cyclosporine analogues (e.g., CRV431) have been described in PCT application published as WO 2020/112562, the content of which is incorporated herein by reference in its entirety.

In some embodiments, the system comprises Vitamin E, Maisine® CC, propylene glycol, Transcutol®, ethanol, and Cremophor® RH40. The weight ratios of non-cyclosporine analogue components in the system can be different in different embodiments. In some embodiments, the weight ratio of a non-cyclosporine analogue component (e.g., Vitamin E, Maisine® CC, propylene glycol, Transcutol®, ethanol, or Cremophor® RH40) relative to another non-cyclosporine analogy components (e.g., Vitamin E, Maisine® CC, propylene glycol, Transcutol®, ethanol, or Crenophor® RH40) in the system can be between about 0.1 and about 10. In some embodiments, the weight ratio of a non-cyclosporine analogue component relative to another non-cyclosporine analogue component (or relative to all other non-cyclosporine analogue components) in the system can be between about 0.1 and about 10. In some embodiments, the weight ratio of a non-cyclosporine analogue component relative to another non-cyclosporine analogy component (or relative to all other non-cyclosporine components) in the system can be, be about, be at least, be at least about, be at most, or be at most about, 0.01:1, 0.015:1, 0.02:1, 0.025:1, 0.03:1, 0.035:1, 0.04:1, 0.045:1, 0.05:1, 0.055:1, 0.06:1, 0.065:1, 0.07:1, 0.075:1, 0.08:1, 0.085:1, 0.09:1, 0.095:1, 0.1:1, 0.15:1, 0.2:1, 0.25:1, 0.3:1, 0.35:1, 0.4:1, 0.45:1, 0.5:1, 0.55:1, 0.6:1, 0.65:1, 0.7:1, 0.75:1, 0.8:1, 0.85:1, 0.9:1, 0.95:1, 1:1, 1.05:1, 1.1:1, 1.15:1, 1.2:1, 1.25:1, 1.3:1, 1.35:1, 1.4:1, 1.45:1, 1.5:1, 1.55:1, 1.6:1, 1.65:1, 1.7:1, 1.75:1, 1.8:1, 1.85:1, 1.9:1, 1.95:1, 2:1, 2.05:1, 2.1:1, 2.15:1, 2.2:1, 2.25:1, 2.3:1, 2.35:1, 2.4:1, 2.45:1, 2.5:1, 2.55:1, 26:1, 2.65:1, 2.7:1, 2.75:1, 2.8:1, 2.85:1, 2.9:1, 2.95:1, 3:1, 3.05:1, 3.1:1, 3.15:1, 3.2:1, 3.25:1, 3.3:1, 3.35:1, 3.4:1, 3.45:1, 3.5:1, 3.55:1, 3.6:1, 3.65:1, 3.7:1, 3.75:1, 3.8:1, 3.85:1, 3.9:1, 3.95:1, 4:1, 4.05:1, 4.1:1, 4.15:1, 4.2:1, 4.25:1, 4.3:1, 4.35:1, 4.4:1, 4.45:1, 4.5:1, 4.55:1, 4.6:1, 4.65:1, 4.7:1, 4.75:1, 4.8:1, 4.85:1, 4.9:1, 4.95:1, 5:1, 5.05:1, 5.1:1, 5.15:1, 5.2:1, 5.25:1, 5.3:1, 5.35:1, 5.4:1, 5.45:1, 5.5:1, 5.55:1, 5.6:1, 5.65:1, 5.7:1, 5.75:1, 5.8:1, 5.85:1, 5.9:1, 5.95:1, 6:1, 6.05:1, 6.1:1, 6.15:1, 6.2:1, 6.25:1, 6.3:1, 6.35:1, 6.4:1, 6.45:1, 6.5:1, 6.55:1, 6.6:1, 6.65:1, 6.7:1, 6.75:1, 6.8:1, 6.85:1, 6.9:1, 6.95:1, 7:1, 7.05:1, 7.1:1, 7.15:1, 7.2:1, 7.25:1, 7.3:1, 7.35:1, 7.4:1, 7.45:1, 7.5:1, 7.55:1, 7.6:1, 7.65:1, 7.7:1, 7.75:1, 7.8:1, 7.85:1, 7.9:1, 7.95:1, 8:1, 8.05:1, 8.1:1, 8.15:1, 8.2:1, 8.25:1, 8.3:1, 8.35:1, 8.4:1, 8.45:1, 8.5:1, 8.55:1, 8.6:1, 8.65:1, 8.7:1, 8.75:1, 8.8:1, 8.85:1, 8.9:1, 8.95:1, 9:1, 9.05:1, 9.1:1, 9.15:1, 9.2:1, 9.25:1, 9.3:1, 9.35:1, 9.4:1, 9.45:1, 9.5:1, 9.55:1, 9.6:1, 9.65:1, 9.7:1, 9.75:1, 9.8:1, 9.85:1, 9.9:1, 9.95:1, 10:1, 10.5:1, 11:1, 11.5:1, 12:1, 12.5:1, 13:1, 13.5:1, 14:1, 14.5:1, 15:1, 15.5:1, 16:1, 16.5:1, 17:1, 17.5:1, 18:1, 18.5:1, 19:1, 19.5:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, or number or a range between any two of these values. In some embodiments, the system comprises Vitamin E, Maisine® CC, propylene glycol. Transcutol®, ethanol, and Cremophor® RH40 at a weight ratio of 1/1/5/52.4/4 to 1/1.5/2.5/5/2.4/5. In some embodiments, the system comprises Vitamin E, Maisine® CC, propylene glycol, Transcutol®, ethanol, and Cremophor® RH40 at a weight ratio of (0.75-1.5)/(0.5-2)/(2-5)/(2-5)/(2-2.4)/(4-8).

In some embodiments, the SMEDD comprises the cyclosporine analogue (e.g., CRV431) at a concentration of from about 10 mg/mL to about 90 mg/mL, such as 10 mg/mL, 20 mg/mL, 30 mg/mL, 40 mg/mL, 50 mg/mL, 60 mg/mL, 70 mg/mL, 80 mg/mL, 90 mg/mL, a range between any two of these values, or any value within 10 mg/mL to 90 mg/mL. In some embodiments, the system comprises the cyclosporine analogue (e.g., CRV431) at a concentration of about 90 mg/mL. In some embodiments, the system comprises the cyclosporine analogue (e.g., CRV431) at a concentration of, or of about, 70 mg/mL.

The composition is a pharmaceutical composition comprising a cyclosporine analogue (e.g., CRV431), or a pharmaceutically acceptable salt, solvate, or stereoisomer thereof, and one or more pharmaceutically acceptable excipients. In some embodiments, the composition is administered to the subject by intravenous administration, nasal administration, pulmonary administration, oral administration, or parenteral administration. In some embodiments, the composition is in the form of powder, pill, tablet, microtablet, pellet, micropellet, capsule, capsule containing microtablets, stable self-microemulsifying drug delivery system (“SMEDD”), liquid, aerosols, suspension, or nanoparticles. In some embodiments, the composition is administered to the subject once, twice, or three times a day. In some embodiments, the composition is administered to the subject once or twice in an emergency situation (e.g., in an ongoing surgery). In some embodiments, the composition is administered to the subject over the course of at least a day, at least two days, at least three days, at least a week, or more. In some embodiments, the composition is administered to the subject at an effective daily dose of the cyclosporine analogue (e.g., CRV431) or a pharmaceutically acceptable salt, solvate, or stereoisomer thereof at from 10 mg to 250 ng.

The therapeutically effective amount and the frequency of administration of, and the length of treatment with, the cyclosporine analogue (e.g. CRV431) may depend on various factors, including the nature and the severity of the thromboembolic disorder, the potency of the cyclosporine analogue (e.g., CRV431), the mode of administration, the age, the body weight, the general health, the gender and the diet of the subject, and the response of the subject to the treatment, and can be determined by the treating physician. In some embodiments, a therapeutically effective amount of the cyclosporine analogue (e.g., CRV431) for treating or preventing a thromboembolic disorder, for reducing or preventing thrombi formation, and/or for reducing or inhibiting procoagulant platelet formation as described herein, is, or is about, 0.1-200 mg, 0.1-50 mg, 0.1-100 mg, 0.1-50 mg, 0.1-30 mg, 0.5-20 mg, 0.5-10 mg or 1-10 mg (e.g., per day or per dose), or as deemed appropriate by the treating physician, which can be administered in a single dose or in divided doses. In certain embodiments, the therapeutically or prophylactically effective dose (e.g., per day or per dose) of the cyclosporine analogue (e.g., CRV431) for treating or preventing a thromboembolic disorder, for reducing or preventing thrombi formation, and/or for reducing or inhibiting procoagulant platelet formation as described herein, is, or is about, 0.1-1 mg (e.g., 0.1 mg, 0.5 mg or 1 mg), 1-5 mg (e.g., 1 mg, 2 mg, 3 mg, 4 mg or 5 mg), 5-10 mg (e.g., 5 mg, 6 mg, 7 mg, 8 mg, 9 mg or 10 mg), 10-20 mg (e.g., 10 mg, 15 mg or 20 mg), 20-30 mg (e.g., 20 mg, 25 mg or 30 mg), 30-40 mg (e.g., 30 mg, 35 mg or 40 mg), 40-50 mg (e.g., 40 mg, 45 mg or 50 mg), 50-100 mg (e.g., 50 mg, 60 mg, 70 mg, 80 mg, 90 mg or 100 mg), 100-150 mg (e.g., 100 rug, 125 mg or 150 mg), or 150-200 mg (e.g., 150 ng, 175 mg or 200 mg). In some embodiments, the therapeutically or prophylactically effective dose (e.g., per day or per dose) of the cyclosporine analogue (e.g., CRV431) for treating or preventing a thromboembolic disorder, for reducing or preventing thrombi formation, and/or for reducing or inhibiting procoagulant platelet formation as described herein, is, or is about, 1 mg/kg, 11 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2 mg/kg, 2.1 mg/kg, 2.2 mg/kg, 2.3 mg/kg, 2.4 mg/kg, 2.5 mg/kg, 2.6 mg/kg, 2.7 mg/kg, 2.8 mg/kg, 2.9 mg/kg, 3 mg/kg, 3.1 mg/kg, 3.2 mg/kg, 3.3 mg/kg, 3.4 mg/kg, 3.5 mg/kg, 3.6 mg/kg, 3.7 mg/kg, 3.8 mg/kg, 3.9 mg/kg, 4 mg/kg, 4.1 mg/kg, 4.2 mg/kg, 4.3 mg/kg, 4.4 mg/kg, 4.5 mg/kg, 4.6 mg/kg, 4.7 mg/kg, 4.8 mg/kg, 4.9 mg/kg, 5 mg/kg, 6 mg/kg, 7 mg/kg, 8 mg/kg, 9 mg/kg, 10 mg/kg, 11 mg/kg, 12 mg/kg, 13 mg/kg, or a number or a range between any two of these values. In some embodiments, the therapeutically or prophylactically effective dose of the cyclosporine analogue (e.g., CRV431) is administered one or more (e.g., two, three or more) times a day, or once every two or three days, or once, twice or thrice a week, or as deemed appropriate by the treating physician. In some embodiments, the composition comprises a therapeutically or prophylactically effective amount of the cyclosporine analogue (e.g., CRV431) or a pharmaceutically acceptable salt, solvate, or stereoisomer thereof.

The cyclosporine analogue (e.g., CRV431) can also be dosed in an irregular manner. For example, the cyclosporine analogue (e.g., CRV431) can be administered once, twice or thrice in a period of 30 minutes, one hour, two hours or more, in an irregular manner. Furthermore, the cyclosporine analogue (e.g., CRV431) can be taken pro re rata (as needed). For instance, the cyclosporine analogue (e.g., CRV431) can be administered 1, 2, 3, 4, 5 or more times, whether in a regular or irregular manner, until the thromboembolic disorder/condition improves. Once relief from the thromboembolic disorder/condition is achieved, dosing of the cyclosporine analogue (e.g., CRV431) can optionally be discontinued. If the thromboembolic disorder/condition returns, administration of the cyclosporine analogue (e.g., CRV431), whether in a regular or irregular manner, can be resumed. The appropriate dosage of, frequency of dosing of and length of treatment with the cyclosporine analogue (e.g., CRV431) can be determined by the treating physician.

The cyclosporine analogue (e.g., CRV431) can also be used prophylactically to treat or prevent a thromboembolic disorder, or to prevent or reduce formation of thrombi, or to reduce or inhibit procoagulant platelet formation. The prophylactically effective amount of a cyclosporine analogue (e.g., CRV431) can be any therapeutically effective amount of the cyclosporine analogue (e.g., CRV431) described herein.

The cyclosporine analogue (e.g., CRV431) can be administered via any suitable route. Potential routes of administration of the cyclosporine analogue (e.g., CRV431) include without limitation oral, parenteral (including intramuscular, subcutaneous, intradermal, intravascular, intravenous, intraarterial, intramedullary and intrathecal), intracavitary, intraperitoneal, and topical (including dermal/epicutaneous, transdermal, mucosal, transmucosal, intranasal [e.g., by nasal spray or drop], intraocular [e.g., by eye drop], pulmonary [e.g., by oral or nasal inhalation], buccal, sublingual, rectal and vaginal), In certain embodiments, the cyclosporine analogue (e.g., CRV431) is administered orally (e.g., as a capsule or tablet, optionally with an enteric coating). In other embodiments, the cyclosporine analogue (e.g., CRV431) is administered parenterally (e.g., intravenously, subcutaneously or intradermally). In further embodiments, the cyclosporine analogue (e.g., CRV431) is administered topically (e.g., dermally/epicutaneously, transdermally, mucosally, transmucosally, buccally or sublingually).

In additional embodiments, the cyclosporine analogue (e.g., CRV431) is administered without food. In some embodiments, the cyclosporine analogue (e.g., CRV431) is administered at least about 1 or 2 hours before or after a meal. In certain embodiments, the cyclosporine analogue (e.g., CRV431) is administered at least about 2 hours after an evening meal. The cyclosporine analogue (e.g., CRV431) can also be taken substantially concurrently with food (e.g., within about 0.5, 1 or 2 hours before or after a meal, or with a meal).

In some embodiments where a more rapid establishment of a therapeutic level of the cyclosporine analogue (e.g., CRV431) is desired, the cyclosporine analogue (e.g., CRV431) is administered under a dosing schedule in which a loading dose is administered, followed by (i) one or more additional loading doses and then one or more therapeutically effective maintenance doses, or (ii) one or more therapeutically effective maintenance doses without an additional loading dose, as deemed appropriate by the treating physician. A loading dose of a drug is typically larger (e.g., about 1.5, 2, 3, 4 or 5 times larger) than a subsequent maintenance dose and is designed to establish a therapeutic level of the drug more quickly. The one or more therapeutically effective maintenance doses can be any therapeutically effective dose described herein. In certain embodiments, the loading dose is about three times greater than the maintenance dose. In some embodiments, a loading dose of the cyclosporine analogue (e.g., CRV431) is administered, followed by administration of a maintenance dose of the cyclosporine analogue (e.g., CRV431) after an appropriate time (e.g., after about 12 or 24 hours) and thereafter for the duration of therapy—e.g., a loading dose of the cyclosporine analogue (e.g., CRV431) is administered on day 1 and a maintenance dose is administered on day 2 and thereafter for the duration of therapy. In some embodiments, the cyclosporine analogue (e.g., CRV431) is administered in a loading, dose of about 1.5, 3, 15 or 30 mg (e.g., 3×about 0.5, 1, 5 or 10 mg) orally (e.g., as a tablet) on day 1, followed by a maintenance dose of about 0.5, 1, 5 or 10 mg orally (e.g., as a tablet) once daily, optionally at bedtime, for at least about 2 weeks, 1 month (4 weeks), 6 weeks, 2 months, 10 weeks, 3 months, 4 months, 5 months, 6 months, 1 year, 1.5 years, 2 years, 3 years or longer (e.g., at least about 6 weeks, 2 months, 3 months or 6 months). In certain embodiments, the cyclosporine analogue (e.g., CRV431) is administered in a loading dose of about 15 mg (e.g., 3×about 5 mg) orally (e.g., as a tablet) on day 1, followed by a maintenance dose of about 5 mg orally (e.g., as a tablet) once daily, optionally at bedtime, for at least about 2 weeks, 1 month, 6 weeks, 2 months, 3 months, 6 months, 1 year, 1.5 years, 2 years, 3 years or longer (e.g., at least about 6 weeks, 2 months, 3 months or 6 months).

In some embodiments, a first loading dose of the cyclosporine analogue (e.g., CRV431) is administered on day 1, a second loading dose is administered on day 2, and a maintenance dose is administered on day 3 and thereafter for the duration of therapy. In certain embodiment, the first loading dose is about three times greater than the maintenance dose, and the second loading dose is about two times greater than the maintenance dose.

As disclosed herein, the therapeutic agent (e.g., the cyclosporine analogue (e.g., CRV431)) can be formulated for administration in a pharmaceutical composition comprising a physiologically acceptable surface active agents, carriers, diluents, excipients, smoothing agents, suspension agents, film forming substances, coating assistants, or a combination thereof. In some embodiments, the therapeutic agent (e.g., the cyclosporine analogue (e.g., CRV431)) are formulated for administration with a pharmaceutically acceptable carrier or diluent. The therapeutic agent (e.g., the cyclosporine analogue (e.g., CRV431)) can be formulated as a medicament with a standard pharmaceutically acceptable carrier(s) and/or excipient(s) as is routine in the pharmaceutical art. The exact nature of the formulation will depend upon several factors including the desired route of administration, in some embodiments, the cyclosporine analogue (e.g., CRV431) is formulated for oral, intravenous, intragastric, intravascular or intraperitoneal administration. Standard pharmaceutical formulation techniques may be used, such as those disclosed in Remington's The Science and Practice of Pharmacy, 21st Ed, Lippincott Williams & Wilkins (2005), incorporated herein by reference in its entirety.

The term “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. In addition, various adjuvants such as are commonly used in the art may be included. Considerations for the inclusion of various components in pharmaceutical compositions are described, e.g., in Gilman et al. (Eds.) (1990); Goodman and Gilman's: The Pharmacological Basis of Therapeutics, 8th Ed., Pergamon Press, which is incorporated herein by reference in its entirety.

Some examples of substances, which can serve as pharmaceutically-acceptable carriers or components thereof, are sugars, such as lactose, glucose and sucrose: starches, such as corn starch and potato starch; cellulose and its derivatives, such as sodium carboxymethyl cellulose, powdered tragacanth; malt, gelatin; talc; solid lubricants, such as stearic acid and magnesium stearate; calcium sulfate; vegetable oils, such as peanut oil, cottonseed oil, sesame oil, olive oil, con oil and theobroma oil, polyols such as propylene glycol, glycerin, sorbitol, mannitol, and polyethylene glycol, alginic acid; emulsifiers, such as the TWEENS; wetting agents, such sodium lauryl sulfate; coloring agents; flavoring agents; tableting agents, stabilizers; antioxidants; preservatives; pyrogen-free water; isotonic saline; and phosphate buffer solutions.

The choice of a pharmaceutically-acceptable carrier to be used in conjunction with the subject therapeutic agent is basically determined by the way the composition is to be administered.

The compositions described herein are preferably provided in unit dosage form. As used herein, a “unit dosage form” is a composition containing an amount of a therapeutic agent (e.g., a cyclosporine analogue (e.g., CRV431)) that is suitable for administration to an animal, preferably mammal subject, in a single dose, according to good medical practice. The preparation of a single or unit dosage form however, does not imply that the dosage form is administered once per day or once per course of therapy. Such dosage forms are contemplated to be administered once, twice, thrice or more per day and may be administered as infusion over a period of time (e.g., from about 30 minutes to about 2-6 hours), or administered as a continuous infusion, and can be given more than once during a course of therapy, though a single administration is not specifically excluded. The skilled artisan will recognize that the formulation does not specifically contemplate the entire course of therapy and such decisions are left for those skilled in the art of treatment rather than formulation.

The compositions useful as described above may be in any of a variety of suitable forms for a variety of routes for administration, for example, for oral, nasal, rectal, topical (including transdermal), ocular, intracerebral, intracranial, intrathecal, intra-arterial, intravenous, intramuscular, or other parental routes of administration. The skilled artisan will appreciate that oral and nasal compositions include compositions that are administered by inhalation, and made using available methodologies. Depending upon the particular route of administration desired, a variety of pharmaceutically-acceptable carriers well-known in the art may be used. Pharmaceutically-acceptable carriers include, for example, solid or liquid fillers, diluents, hydrotropies, surface-active agents, and encapsulating substances. Optional pharmaceutically-active materials may be included, which do not substantially interfere with the inhibitory activity of the therapeutic agent (e.g., the cyclosporine analogue (e.g., CRV43)). The amount of carrier employed in conjunction with the therapeutic agent (e.g., the cyclosporine analogue (e.g., CRV431)) is sufficient to provide a practical quantity of material for administration per unit dose of the therapeutic agent (e.g., the cyclosporine analogue (e.g., CRV431)). Techniques and compositions for making dosage forms useful in the methods described herein are described in the following references, ail incorporated by reference herein: Modern Pharmaceutics, 4th Ed, Chapters 9 and 10 (Banker & Rhodes, editors, 2002); Lieberman et αi., Pharmaceutical Dosage Forms: Tablets (1989), and Ansel, Introduction to Pharmaceutical Dosage Forms 8th Edition (2004).

Various oral dosage forms can be used, including such solid forms as tablets, capsules, and granules. Tablets can be compressed, tablet triturates, enteric-coated, sugar-coated, film-coated, or multiple-compressed, containing suitable binders, lubricants, diluents, disintegrating agents, coloring agents, flavoring agents, flow-inducing agents, and melting agents. Liquid oral dosage forms include aqueous solutions, emulsions, suspensions, solutions and/or suspensions reconstituted from non-effervescent granules, and effervescent preparations reconstituted from effervescent granules, containing suitable solvents, preservatives, emulsifying agents, suspending agents, diluents, sweeteners, melting agents, coloring agents and flavoring agents.

The pharmaceutically-acceptable carriers suitable for the preparation of unit dosage forms for peroral administration is well-known in the art. Tablets typically comprise conventional pharmaceutically-compatible adjuvants as inert diluents, such as calcium carbonate, sodium carbonate, mannitol, lactose and cellulose; binders such as starch, gelatin and sucrose; disintegrants such as starch, alginic acid and croscarmellose; lubricants such as magnesium stearate, stearic acid and talc. Glidants such as silicon dioxide can be used to improve flow characteristics of the powder mixture. Coloring agents, such as the FD&C dyes, can be added for appearance. Sweeteners and flavoring agents, such as aspartame, saccharin, menthol, peppermint, and fruit flavors, are useful adjuvants for chewable tablets. Capsules typically comprise one or more solid diluents disclosed above. The selection of carrier components depends on secondary considerations like taste, cost, and shelf stability, which are not critical, and can be readily made by a person skilled in the art.

Peroral compositions also include liquid solutions, emulsions, suspensions, and the like. The pharmaceutically acceptable carriers suitable for preparation of such compositions are well known in the art. Typical components of carriers for syrups, elixirs, emulsions and suspensions include ethanol, glycerol, propylene glycol, polyethylene glycol, liquid sucrose, sorbitol and water. For a suspension, typical suspending agents include sodium carboxymethyl cellulose, AVICEL RC-591, tragacanth and sodium alginate; typical wetting agents include lecithin and polysorbate 80; and typical preservatives include methyl paraben and sodium benzoate. Peroral liquid compositions may also contain one or more components such as sweeteners, flavoring agents and colorants disclosed above.

Other compositions useful for attaining systemic delivery of the subject therapeutic agents include sublingual, buccal and nasal dosage forms, Such compositions typically comprise one or more of soluble filler substances such as sucrose, sorbitol and mannitol; and binders such as acacia, microcrystalline cellulose, carboxymethyl cellulose and hydroxypropyl methyl cellulose. Glidants, lubricants, sweeteners, colorants, antioxidants and flavoring agents disclosed above may also be included.

For topical use, creams, ointments, gels, solutions or suspensions, etc., containing the therapeutic agent (e.g., the cyclosporine analogue (e.g., CRV431)) disclosed herein are employed. Topical formulations may generally be comprised of a pharmaceutical carrier, co-solvent, emulsifier, penetration enhancer, preservative system, and emollient.

For intravenous administration, the therapeutic agent (e.g., the cyclosporine analogue (e.g., CRV431)) and compositions described herein may be dissolved or dispersed in a pharmaceutically acceptable diluent, such as a saline or dextrose solution. Suitable excipients may be included to achieve the desired pH, including but not limited to NaOH, sodium carbonate, sodium acetate, HCl, and citric acid. In various embodiments, the pH of the final composition ranges from 2 to 8, or preferably from 4 to 7. Antioxidant excipients may include sodium bisulfite, acetone sodium bisulfite, sodium formaldehyde, sulfoxylate, thiourea, and EDTA. Other non-limiting examples of suitable excipients found in the final intravenous composition may include sodium or potassium phosphates, citric acid, tartaric acid, gelatin, and carbohydrates such as dextrose, mannitol, and dextran. Further acceptable excipients are described in Powell, et al., Compendium of Excipients for Parenteral Formulations, PDA J Pharm Sci and Tech 1998, 52 238-31 1 and Nema et al., Excipients and Their Role in Approved Injectable Products. Current Usage and Future Directions, PDA J Pharm Sci and Tech 2011, 65 287-332, both of which are incorporated herein by reference in their entirety. Antimicrobial agents may also be included to achieve a bacteriostatic or fungistatic solution, including but not limited to phenyl mercuric nitrate, thimerosal, benzethonium chloride, benzalkonium chloride, phenol, cresol, and chlorobutanol.

The compositions for intravenous administration may be provided to caregivers in the form of one more solids that are reconstituted with a suitable diluent such as sterile water, saline or dextrose in water shortly prior to administration. In other embodiments, the compositions are provided in solution ready to administer parenterally. In still other embodiments, the compositions are provided in a solution that is further diluted prior to administration. In embodiments that include administering a combination of a therapeutic agent (e.g., the cyclosporine analogue (e.g., CRV431)) described herein and another agent, the combination may be provided to caregivers as a mixture, or the caregivers may mix the two agents prior to administration, or the two agents may be administered separately.

In non-human animal studies, applications of potential products are commenced at higher dosage levels, with dosage being decreased until the desired effect is no longer achieved or adverse side effects disappear. The dosage may range broadly, depending upon the desired effects and the therapeutic indication. Typically, dosages may be between about 0.1 mg/kg and 4000 mg/kg body weight, preferably between about 80 mg/kg and 1600 mg/kg body weight. Alternatively, dosages may be based and calculated upon the surface area of the patient, as understood by those of skill in the art.

Depending on the severity and responsiveness of the condition to be treated, dosing can also be a single administration of a slow release composition, with course of treatment lasting from several days to several weeks or until cure is effected or diminution of the disease state is achieved. The amount of a composition to he administered will, of course, be dependent on many factors including the subject being treated, the severity of the affliction, the manner of administration, the judgment of the prescribing physician. The therapeutic agent (e.g., cyclosporine analogue (e.g., CRV431)) or combination of therapeutic agents disclosed herein may be administered orally or via injection at a dose from 0, 1 mg/kg to 4000 mg/kg of the patient's body weight per day. The dose range for adult humans is generally from 1 g to 100 g/day. Tablets or other forms of presentation provided in discrete units may conveniently contain an amount of the therapeutic agent (e.g., cyclosporine analogue (e.g., CRV431)) or combination of therapeutic agents disclosed herein which is effective at such dosage or as a multiple of the same, for instance, units containing 1 g to 60 g (for example, from about 5 g to 20 g, from about 10 g to 50 g, from about 20 g to 40 g, or from about 25 g to 35 g). The precise amount of therapeutic agent administered to a patient will be the responsibility of the attendant physician. However, the dose employed will depend on a number of factors, including the age and sex of the patient, the precise disorder being treated, and its severity. Additionally, the route of administration may vary depending on the condition and its severity. A typical dose of the therapeutic agent (e.g., cyclosporine analogue (e.g., CRV431)) can be from 0.02 g to 1.25 g per kg of body weight, for example from 0.1 g to 0.5 g per kg of body weight, depending on such parameters. In some embodiments, a dosage of the therapeutic agent (e.g., cyclosporine analogue (e.g., CRV431)) can be from 1 g to 100 g, for example, from 10 g to 80 g, from 15 g to 60 g, from 20 g to 40 g, or from 25 g to 35 g. In A physician will be able to determine the required dosage of the therapeutic agent (e.g., cyclosporine analogue (e.g., CRV431)) for any particular subject.

The exact formulation, route of administration and dosage for the pharmaceutical compositions of the therapeutic agent (e.g., cyclosporine analogue (e.g., CRV431)) or combination of therapeutic agents disclosed herein can be chosen by the individual physician in view of the patient's condition. Typically, the dose range of the composition administered to the patient can be from about 0.1 to about 4000 mg/kg of the patient's body weight. The dosage may be a single one or a series of two or more given in the course of one or more days, as is needed by the patient. In instances where human dosages for therapeutic agents have been established for at least some condition, the present disclosure will use those same dosages, or dosages that are between about 0.1% and about 5000%, more preferably between about 25% and about 1000% of the established human dosage. Where no human dosage is established, as will be the case for newly-discovered pharmaceutical compounds, a suitable human dosage can be inferred from ED₅₀ or ID₅₀ values, or other appropriate values derived from in vitro or in vivo studies, as qualified by toxicity studies and efficacy studies in animals.

It should be noted that the attending physician would know how to and when to terminate, interrupt, or adjust administration due to toxicity or organ dysfunctions. Conversely, the attending physician would also know to adjust treatment to higher levels if the clinical response were not adequate (precluding toxicity). The magnitude of an administrated dose in the management of the disorder of interest will vary with the severity of the condition to be treated and to the route of administration. The severity of the condition may, for example, be evaluated, in part, by standard prognostic evaluation methods. Further, the dose and perhaps dose frequency, will also vary according to the age, body weight, and response of the individual patient. A program comparable to that discussed above may be used in veterinary medicine.

Although the exact dosage will be determined on a drug-by-drug basis, in most cases, some generalizations regarding the dosage can be made. In cases of administration of a pharmaceutically acceptable salt, dosages may be calculated as the free base. In some embodiments, the composition is administered 1 to 4 times per day. Alternatively the compositions disclosed herein may be administered by continuous intravenous infusion, e.g., at a dose of each active ingredient up to 100 g per day. As will be understood by those of skill in the art, in certain situations it may be necessary to administer the compositions disclosed herein in amounts that exceed, or even far exceed, the above-stated, preferred dosage range in order to effectively and aggressively treat particularly aggressive diseases or infections. In some embodiments, the therapeutic agent (e.g., cyclosporine analogue (e.g., CRV431)) or combination of therapeutic agents disclosed herein will be administered for a period of continuous therapy, for example for a week or more, or for months or years.

In some embodiments, the dosing regimen of the therapeutic agent (e.g., cyclosporine analogue (e.g., CRV431)) or combination of therapeutic agents disclosed herein is administered for a period of time, which time period can be, for example, from at least about 1 week to at least about 4 weeks, from at least about 4 weeks to at least about 8 weeks, from at least about 4 weeks to at least about 12 weeks, from at least about 4 weeks to at least about 16 weeks, or longer. The dosing regimen of the therapeutic agent (e.g., cyclosporine analogue (e.g., CRV431)) or combination of therapeutic agents disclosed herein can be administered three times a day, twice a day, daily, every other day, three times a week, every other week, three times per month, once monthly, substantially continuously or continuously.

The cyclosporine analogue (e.g., CRV431) can be administered alone or in the form of a composition (e.g., a pharmaceutical composition). In some embodiments, a pharmaceutical composition comprises a cyclosporine analogue (e.g., CRV431) or a pharmaceutically acceptable salt, solvate, hydrate, clathrate, polymorph, prodrug or metabolite thereof, and one or more pharmaceutically acceptable carriers or excipients. The composition can optionally contain one or more additional therapeutic agents as described herein. A pharmaceutical composition contains a therapeutically effective amount of a therapeutic agent (e.g., a cyclosporine analogue (e.g., CRV431)) and one or more pharmaceutically acceptable carriers or excipients, and is formulated for administration to a subject for therapeutic use. For purposes of the content of a pharmaceutical composition, the terms “therapeutic agent”, “active ingredient”, “active agent” and “drug” encompass prodrugs.

A pharmaceutical composition contains a therapeutic agent (e.g., a cyclosporine analogue (e.g., CRV431)) in substantially pure form. In some embodiments, the purity of the therapeutic agent is at least about 95%, 96%, 97%, 98% or 99%. In certain embodiments, the purity of the therapeutic agent is at least about 98% or 99%. In addition, a pharmaceutical composition is substantially free of contaminants or impurities. In some embodiments, the level of contaminants or impurities other than residual solvent in a pharmaceutical composition is no more than about 5%, 4%, 3%, 2% or 1% relative to the combined weight of the intended active and inactive ingredients. In certain embodiments, the level of contaminants or impurities other than residual solvent in a pharmaceutical composition is no more than about 2% or 1% relative to the combined weight of the intended active and inactive ingredients. Pharmaceutical compositions generally are prepared according to current good manufacturing practice (GMP), as recommended or required by, e.g., the Federal Food, Drug, and Cosmetic Act § 501(a)(2)(B) and the International Conference on Harmonisation Q7 Guideline.

Pharmaceutically acceptable carriers and excipients include pharmaceutically acceptable materials, vehicles and substances. Non-limiting examples of excipients include liquid and solid fillers, diluents, binders, lubricants, glidants, solubilizers, surfactants, dispersing agents, disintegration agents, emulsifying agents, wetting agents, suspending agents, thickeners, solvents, isotonic agents, buffers, pH adjusters, stabilizers, preservatives, antioxidants, antimicrobial agents, antibacterial agents, antifungal agents, absorption-delaying agents, sweetening agents, flavoring agents, coloring agents, adjuvants, encapsulating materials and coating materials. The use of such excipients in pharmaceutical formulations is known in the art. For example, conventional vehicles and carriers include without limitation oils (e.g., vegetable oils, such as sesame oil), aqueous solvents (e.g., saline, phosphate-buffered saline [PBS] and isotonic solutions [e.g., Ringer's solution]), and solvents (e.g., dimethyl sulfoxide [DMSO] and alcohols [e.g., ethanol, glycerol and propylene glycol]). Except insofar as any conventional carrier or excipient is incompatible with the active ingredient, the disclosure encompasses the use of conventional carriers and excipients in formulations containing a therapeutic agent (e.g., a cyclosporine analogue (e.g., CRV431)). See, e.g., Remington: The Science and Practice of Pharmacy, 21st Ed, Lippincott Williams & Wilkins (Philadelphia, Pennsylvania [2005]); Handbook of Pharmaceutical Excipients, 5th Ed., Rowe et. al., Eds., The Pharmaceutical Press and the American Pharmaceutical Association (2005); Handbook of Pharmaceutical Additives, 3rd Ed., Ash and Ash, Eds., Gower Publishing Co. (2007); and Pharmaceutical Preformulation and Formulation, Gibson, Ed., CRC Press (Boca Raton, Florida, 2004).

Proper formulation can depend on various factors, such as the mode of administration chosen. Potential modes of administration of pharmaceutical compositions comprising a cyclosporine analogue (e.g., (CRV431) include without limitation oral, parenteral (including intramuscular, subcutaneous, intradermal, intravascular, intravenous, intraarterial, intraperitoneal, intramedullary, intrathecal and topical), intracavitary, and topical (including dermal/epicutaneous, transdermal, mucosal, transmucosal, intranasal [e.g., by nasal spray or drop], pulmonary [e.g., by oral or nasal inhalation], buccal, sublingual, rectal [e.g., by suppository], and vaginal [e.g., by suppository]).

As an example, formulations of a cyclosporine analogue (e.g., CRV431) suitable for oral administration can be presented as, e.g., boluses; tablets, capsules, pills, cachets or lozenges; as powders or granules; as semisolids, electuaries, pastes or gels; as solutions or suspensions in an aqueous liquid or/and a non-aqueous liquid; or as oil-in-water liquid emulsions or water-in-oil liquid emulsions.

Tablets can contain a cyclosporine analogue (e.g., CRV431) in admixture with, e.g., a filler or inert diluent (e.g., calcium carbonate, calcium phosphate, lactose, mannitol or microcrystalline cellulose), a binding agent (e.g., a starch, gelatin, acacia, alginic acid or a salt thereof, or microcrystalline cellulose), a lubricating agent (e.g., stearic acid, magnesium stearate, talc or silicon dioxide), and a disintegrating agent (e.g., crospovidone, croscarmellose sodium or colloidal silica), and optionally a surfactant (e.g., sodium lauryl sulfate). The tablets can be uncoated or can be coated with, e.g., an enteric coating that protects the active ingredient from the acidic environment of the stomach, or with a material that delays disintegration and absorption of the active ingredient in the gastrointestinal tract and thereby provides a sustained action over a longer time period. In certain embodiments, a tablet comprises a cyclosporine analogue (e.g., CRV431), mannitol, microcrystalline cellulose, magnesium stearate, silicon dioxide, croscarmellose sodium and sodium lauryl sulfate, and optionally lactose monohydrate, and the tablet is optionally film-coated (e.g., with Opadry®).

Push-fit capsules or two-piece hard gelatin capsules can contain a cyclosporine analogue (e.g., CRV431) in admixture with, e.g., a filler or inert solid diluent (e.g., calcium carbonate, calcium phosphate, kaolin or lactose), a binder (e.g., a starch), a glidant or lubricant (e.g., talc or magnesium stearate), and a disintegrant (e.g., crospovidone), and optionally a stabilizer or/and a preservative. For soft capsules or single-piece gelatin capsules, a cyclosporine analogue (e.g., CRV431) can be dissolved or suspended in a suitable liquid (e.g., liquid polyethylene glycol or an oil medium, such as a fatty oil, peanut oil, olive oil or liquid paraffin), and the liquid-filled capsules can contain one or more other liquid excipients or/and semi-solid excipients, such as a stabilizer or/and an amphiphilic agent (e.g., a fatty acid ester of glycerol, propylene glycol or sorbitol).

Compositions for oral administration can also be formulated as solutions or suspensions in an aqueous liquid or/and a non-aqueous liquid, or as oil-in-water liquid emulsions or water-in-oil liquid emulsions, Dispersible powder or granules of a cyclosporine analogue (e.g., CRV431) can be mixed with any suitable combination of an aqueous liquid, an organic solvent or/and an oil and any suitable excipients (e.g., any combination of a dispersing agent, a wetting agent, a suspending agent, an emulsifying agent or/and a preservative) to form a solution, suspension or emulsion.

A cyclosporine analogue (e.g., CRV431) can also be formulated for parenteral administration by injection or infusion to circumvent gastrointestinal absorption and first-pass metabolism. A representative parenteral route is intravenous.

Additional advantages of intravenous administration include direct administration of a therapeutic agent into systemic circulation to achieve a rapid systemic effect, and the ability to administer the agent continuously or/and in a large volume if desired. Formulations for injection or infusion can be in the form of, e.g., solutions, suspensions or emulsions in oily or aqueous vehicles, and can contain excipients such as suspending agents, dispersing agents or/and stabilizing agents. For example, aqueous or non-aqueous (e.g., oily) sterile injection solutions can contain a cyclosporine analogue (e.g., CRV431) along with excipients such as an antioxidant, a buffer, a bacteriostat and solutes that render the formulation isotonic with the blood of the subject. Aqueous or non-aqueous sterile suspensions can contain a cyclosporine analogue (e.g., CRV431) along with excipients such as a suspending agent and a thickening agent, and optionally a stabilizer and an agent that increases the solubility of the cyclosporine analogue (e.g., CRV431) to allow for the preparation of a more concentrated solution or suspension. As another example, a sterile aqueous solution for injection or infusion (e.g., subcutaneously or intravenously) can contain a cyclosporine analogue (e.g., CRV431), NaCl, a buffering agent (e.g., sodium citrate), a preservative (e.g., meta-cresol), and optionally a base (e.g., NaOH) or/and an acid (e.g., HCl) to adjust pH.

For topical administration, a cyclosporine analogue (e.g., CRV431) can be formulated as, e.g., a buccal or sublingual tablet or pill. Advantages of a buccal or sublingual tablet or pill include avoidance of first-pass metabolism and circumvention of gastrointestinal absorption. A buccal or sublingual tablet or pill can also be designed to provide faster release of the cyclosporine analogue (e.g., CRV431) for more rapid uptake of it into systemic circulation. In addition to a therapeutically effective amount of the cyclosporine analogue (e.g., CRV431), the buccal or sublingual tablet or pill can contain suitable excipients, including without limitation any combination of fillers and diluents (e.g., mannitol and sorbitol), binding agents (e.g., sodium carbonate), wetting agents (e.g., sodium carbonate), disintegrants (e.g., crospovidone and croscarmellose sodium), lubricants (e.g., silicon dioxide [including colloidal silicon dioxide] and sodium stearyl fumarate), stabilizers (e.g., sodium bicarbonate), flavoring agents (e.g., spearmint flavor), sweetening agents (e.g., sucralose), and coloring agents (e.g., yellow iron oxide).

For topical administration, a cyclosporine analogue (e.g., CRV431) can also be formulated for intranasal administration. The nasal mucosa provides a big surface area, a porous endothelium, a highly vascular subepithelial layer and a high absorption rate, and hence allows for high bioavailability. Moreover, intranasal administration avoids first-pass metabolism and can introduce a significant concentration of the cyclosporine analogue (e.g., CRV431) to the central nervous system, allowing the cyclosporine analogue (e.g., CRV431) to block the central cough reflex via the nucleus tractus solitarius in the cough center in the medulla oblongata, where vagal afferent nerves terminate. An intranasal solution or suspension formulation can comprise a cyclosporine analogue (e.g., CRV431) along with excipients such as a solubility enhancer (e.g., propylene glycol), a humectant (e.g., mannitol or sorbitol), a buffer and water, and optionally a preservative (e.g., benzalkonium chloride), a mucoadhesive agent (e.g., hydroxyethyl cellulose) or/and a penetration enhancer. In certain embodiments, a nasal spray formulation comprises a cyclosporine analogue (e.g., CRV431), microcrystalline cellulose, sodium carboxymethylcellulose, dextrose and water, and optionally an acid (e.g., HCl) to adjust pH. An intranasal solution or suspension formulation can be administered to the nasal cavity by any suitable means, including but not limited to a dropper, a pipette, or spray using, e.g., a metering atomizing spray pump.

An additional mode of topical administration is pulmonary, including by oral inhalation and nasal inhalation, which is described in detail below.

Other suitable topical formulations and dosage forms include without limitation ointments, creams, gels, lotions, pastes and the like, as described in Remington: The Science and Practice of Pharmacy, 21st Ed., Lippincott Williams & Wilkins (Philadelphia, Pennsylvania, 2005).

Ointments are semi-solid preparations that are typically based on petrolatum or a petroleum derivative. Creams are viscous liquids or semi-solid emulsions, either oil-in-water or water-in-oil. Cream bases are water-washable, and contain an oil phase, an emulsifier and an aqueous phase. The oil phase, also called the “internal” phase, generally comprises petrolatum and a fatty alcohol (e.g., cetyl or stearyl alcohol). The aqueous phase typically, although not necessarily, exceeds the oil phase in volume, and usually contains a humectant. The emulsifier in a cream formulation is generally a non-ionic, anionic, cationic or amphoteric surfactant. Gels are semi-solid, suspension-type systems. Single-phase gels contain organic macromolecules (polymers) distributed substantially uniformly throughout the carrier liquid, which is typically aqueous but can also contain an alcohol (e.g., ethanol or isopropanol) and optionally an oil. Lotions are preparations to be applied to the skin surface without friction, and are typically liquid or semi-liquid preparations in which solid particles, including the active agent, are present in a water or alcohol base. Lotions are usually suspensions of finely divided solids and typically contain suspending agents to produce better dispersion as well as compounds useful for localizing and holding the active agent in contact with the skin. Pastes are semi-solid dosage forms in which the active agent is suspended in a suitable base. Depending on the nature of the base, pastes are divided between fatty pastes or those made from single-phase aqueous gels.

Various excipients can be included in a topical formulation. For example, solvents, including a suitable amount of an alcohol, can be used to solubilize the active agent. Other optional excipients include without limitation gelling agents, thickening agents, emulsifiers, surfactants, stabilizers, buffers, antioxidants, preservatives, cooling agents (e.g., menthol), opacifiers, fragrances and colorants. For an active agent having a low rate of permeation through the skin or mucosal tissue, a topical formulation can contain a permeation enhancer to increase the permeation of the active agent through the skin or mucosal tissue. A topical formulation can also contain an irritation-mitigating excipient that reduces any irritation to the skin or mucosa caused by the active agent, the permeation enhancer or any other component of the formulation.

In some embodiments, a cyclosporine analogue (e.g., CRV431) is delivered from a sustained-release composition. As used herein, the term “sustained-release composition” encompasses sustained-release, prolonged-release, extended-release, slow-release and controlled-release compositions, systems and devices. Use of a sustained-release composition can have benefits, such as an improved profile of the amount of the drug or an active metabolite thereof delivered to the target site(s) over a time period, including delivery of a therapeutically effective amount of the drug or an active metabolite thereof over a prolonged time period. In certain embodiments, the sustained-release composition delivers the cyclosporine analogue (e.g., CRV431) over a period of at least about 1 day, 2 days, 3 days, 1 week, 2 weeks, 3 weeks, 1 month, 2 months, 3 months or longer. In some embodiments, the sustained-release composition is a drug-encapsulation system, such as nanoparticles, microparticles or a capsule made of, e.g., a biodegradable polymer or/and a hydrogel. In certain embodiments, the sustained-release composition comprises a hydrogel. Non-limiting examples of polymers of which a hydrogel can be composed include polyvinyl alcohol, acrylate polymers (e.g., sodium poly acrylate), and other homopolymers and copolymers having a relatively large number of hydrophilic groups (e.g., hydroxyl or/and carboxylate groups). In other embodiments, the sustained-release drug-encapsulation system comprises a membrane-enclosed reservoir, wherein the reservoir contains a drug and the membrane is permeable to the drug. Such a drug-delivery system can be in the form of, e.g., a transdermal patch.

In some embodiments, the sustained-release composition is an oral dosage form, such as a tablet or capsule. For example, a drug can be embedded in an insoluble porous matrix such that the dissolving drag must make its way out of the matrix before it can be absorbed through the gastrointestinal tract. Alternatively, a drug can be embedded in a matrix that swells to form a get through which the drug exits. Sustained release can also be achieved by way of a single-layer or multi-layer osmotic controlled-release oral delivery system (OROS). An OROS is a tablet with a semi-permeable outer membrane and one or more small laser-drilled holes in it. As the tablet passes through the body, water is absorbed through the semipermeable membrane via osmosis, and the resulting osmotic pressure pushes the drug out through the hole(s) in the tablet and into the gastrointestinal tract where it can be absorbed.

In further embodiments, the sustained-release composition is formulated as polymeric nanoparticles or microparticles, wherein the polymeric particles can be delivered, e.g., by inhalation or injection or from an implant. In some embodiments, the polymeric implant or polymeric nanoparticles or microparticles are composed of a biodegradable polymer. In certain embodiments, the biodegradable polymer comprises lactic acid or/and glycolic acid [e.g., an L-lactic acid-based copolymer, such as poly(L-lactide-co-glycolide) or poly(L-lactic acid-co-D,L-2-hydroxyoctanoic acid)]. For example, biodegradable polymeric microspheres composed of polylactic acid or/and polyglycolic acid can serve as sustained-release pulmonary drug-delivery systems. The biodegradable polymer of the polymeric implant or polymeric nanoparticles or microparticles can be selected so that the polymer substantially completely degrades around the time the period of treatment is expected to end, and so that the byproducts of the polymer's degradation, like the polymer, are biocompatible.

For a delayed or sustained release of a cyclosporine analogue (e.g., CRV431), a composition can also be formulated as a depot that can be implanted in or injected into a subject, e.g., intramuscularly or subcutaneously. A depot formulation can be designed to deliver the cyclosporine analogue (e.g., CRV431) over a longer period of time, e.g., over a period of at least about 1 week, 2 weeks, 3 weeks, 1 month, 6 weeks, 2 months, 0.3 months or longer. For example, the cyclosporine analogue (e.g., CRV431) can be formulated with a polymeric material (e.g., polyethylene glycol (PEG), polylactic acid (PLA) or polyglycolic acid (PGA), or a copolymer thereof (e.g., PLGA)), a hydrophobic material (e.g., as an emulsion in an oil) and/or an ion-exchange resin, or as a sparingly soluble derivative (e.g., a sparingly soluble salt). As an illustrative example, a cyclosporine analogue (e.g., CRV431) can be incorporated or embedded in sustained-release microparticles composed of PLGA and formulated as a monthly depot.

A cyclosporine analogue (e.g., CRV431) can also be contained or dispersed in a matrix material. The matrix material can comprise a polymer (e.g., ethylene-vinyl acetate) and controls the release of the compound by controlling dissolution or/and diffusion of the compound from, e.g., a reservoir, and can enhance the stability of the compound while contained in the reservoir. Such a release system can be designed as a sustained-release system, can be configured as, e.g., a transdermal or transmucosal patch, and can contain an excipient that can accelerate the compound's release, such as a water-swellable material (e.g., a hydrogel) that aids in expelling the compound out of the reservoir.

The release system can provide a temporally modulated release profile (e.g., pulsatile release) when time variation in plasma levels is desired, or a more continuous or consistent release profile when a constant plasma level is desired. Pulsatile release can be achieved from an individual reservoir or from a plurality of reservoirs. For example, where each reservoir provides a single pulse, multiple pulses (“pulsatile” release) are achieved by temporally staggering the single pulse release from each of multiple reservoirs.

Alternatively, multiple pulses can be achieved from a single reservoir by incorporating several layers of a release system and other materials into a single reservoir. Continuous release can be achieved by incorporating a release system that degrades, dissolves, or allows diffusion of a compound through it over an extended time period. In addition, continuous release can be approximated by releasing several pulses of a compound in rapid succession (“digital” release). An active release system can be used alone or in conjunction with a passive release system, as described in U.S. Pat. No. 5,797,898.

In addition, pharmaceutical compositions comprising a cyclosporine analogue (e.g., CRV431) can be formulated as, e.g., liposomes, micelles (e.g., those composed of biodegradable natural or/and synthetic polymers, such as lactosomes), microspheres, microparticles or nanoparticles, whether or not designed for sustained release.

The pharmaceutical compositions can be manufactured in any suitable manner known in the art, e.g., by means of conventional mixing, dissolving, suspending, granulating, dragee-making, levigating, emulsifying, encapsulating, entrapping or compressing processes.

A pharmaceutical composition can be presented in unit dosage form as a single dose wherein all active and inactive ingredients are combined in a suitable system, and components do not need to be mixed to form the composition to be administered. The unit dosage form can contain an effective dose, or an appropriate fraction thereof, of a therapeutic agent (e.g., a cyclosporine analogue (e.g., CRV431). Representative examples of a unit dosage form include a tablet, capsule or pill for oral administration, and powder in a vial or ampoule for oral or nasal inhalation.

Alternatively, a pharmaceutical composition can be presented as a kit, wherein the active ingredient, excipients and carriers (e.g., solvents) are provided in two or more separate containers (e.g., ampoules, vials, tubes, bottles or syringes) and need to be combined to form the composition to be administered. The kit can contain instructions for storing, preparing and administering the composition (e.g., a solution to be injected intravenously).

A kit can contain all active and inactive ingredients in unit dosage form or the active ingredient and inactive ingredients in two or more separate containers, and can contain instructions for using the pharmaceutical composition.

In some embodiments, a kit contains a cyclosporine analogue (e.g., CRV431) or a pharmaceutically acceptable salt, solvate, hydrate, clathrate, polymorph, prodrug or metabolite thereof, and instructions for administering the compound.

### EXAMPLE

Some aspects of the embodiments discussed above are disclosed in further detail in the following example, which is not in any way intended to limit the scope of the present disclosure.

### Example 1

### Effects gf CRV431 on Platelet Aggregation and Procoagulant Platelet Formation

In the present example, a broad-spectrum cyclophilin inhibitor, CRV431, is evaluated to determine if it can inhibit human platelet aggregation and/or procoagulant platelet formation.

### Methods

Platelet isolation. Blood (40 ml) was obtained from healthy volunteers who had not taken any drugs known to affect platelet function for 2 weeks prior to the study. Prostacyclin-washed platelet suspensions (2.5×10⁸/ml) were prepared in Tyrode's buffer.

Platelet aggregometry. Washed platelets (500 μl/test) were pre-incubated (2 minutes at 37° C.) in a lumi-aggregometer (Chronolog, Havertown, PA, USA) with either vehicle (0.1% DMSO) or CRV431 (0.01-3 μM). Platelet aggregation was then initiated with collagen (1 μg/mL) or thrombin (0.1 U/mL) (Chronolog) and monitored by Aggro-Link software for a further 6 minutes. Extent of platelet aggregation was measured as % Light Transmittance between minutes 2 and 6 and determined by Aggro-Link Software.

Procoagulant platelet formation studies. Washed platelets (100 μl/test) were pre-incubated (2 minutes at 37° C.) with CRV431 (0.01-3 μM) and then activated with combined collagen (1.0 μg/mL) and thrombin (0.1 U/ml), and incubated for a further 10 minutes. Procoagulant platelet formation was assessed by measuring annexin-V-FITC binding to exposed phosphatidylserine (PS) by flow cytometry as described previously. Flow cytometry was performed utilizing a BD Fortessa X20 flow cytometer equipped with a blue (488 nm) laser and 530130 band pass filters to detect FITC. Percent PS-positive platelets were determined post-acquisition using FlowJo software.

Statistics. Statistical analysis was performed using GraphPad Prism 7.0 software. All means are reported with SEM. One-way ANOVA with Dunnet's multiple comparisons test was performed for aggregation studies, and a Repeated-measures One-way ANOVA with Dunnet's multiple comparisons test was performed for procoagulant platelet formation studies. Each N represents an experiment from an independent blood donor. A P-value less than 0.05 was considered significant.

### Results

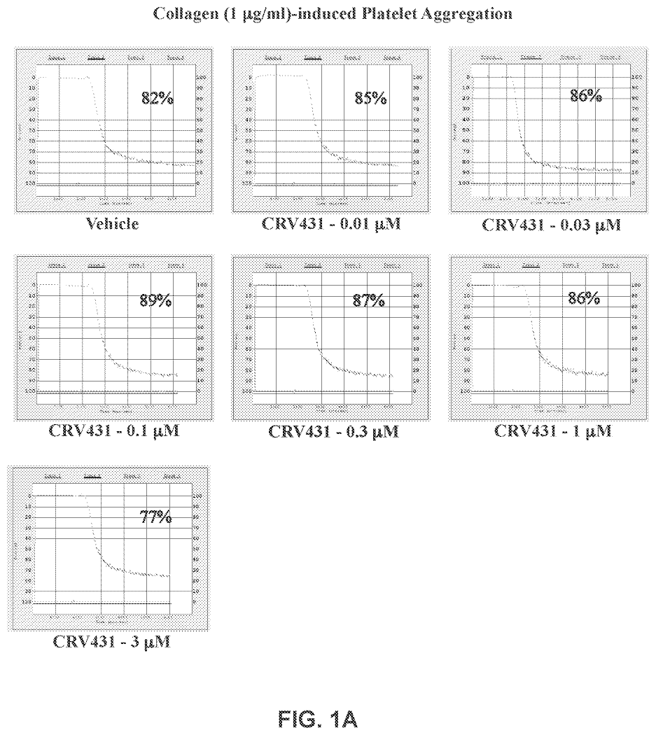

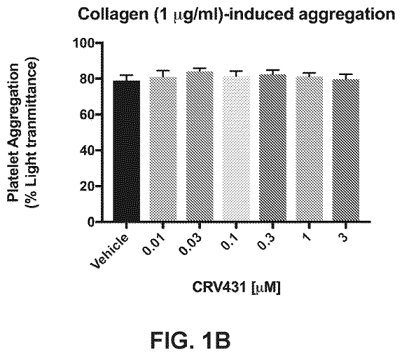

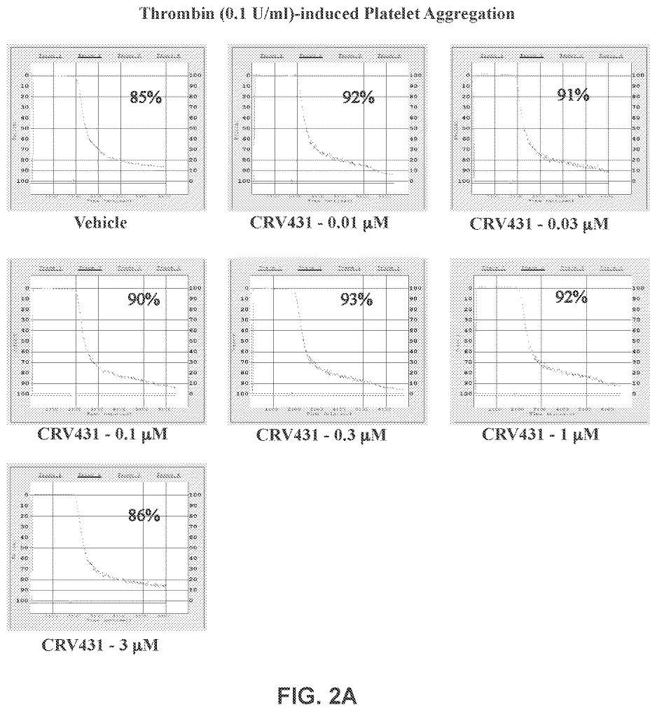

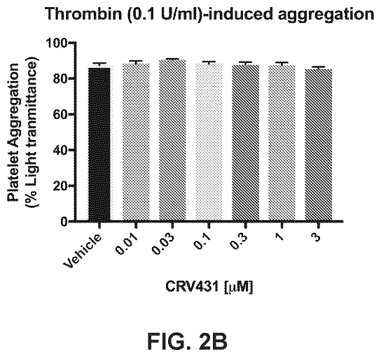

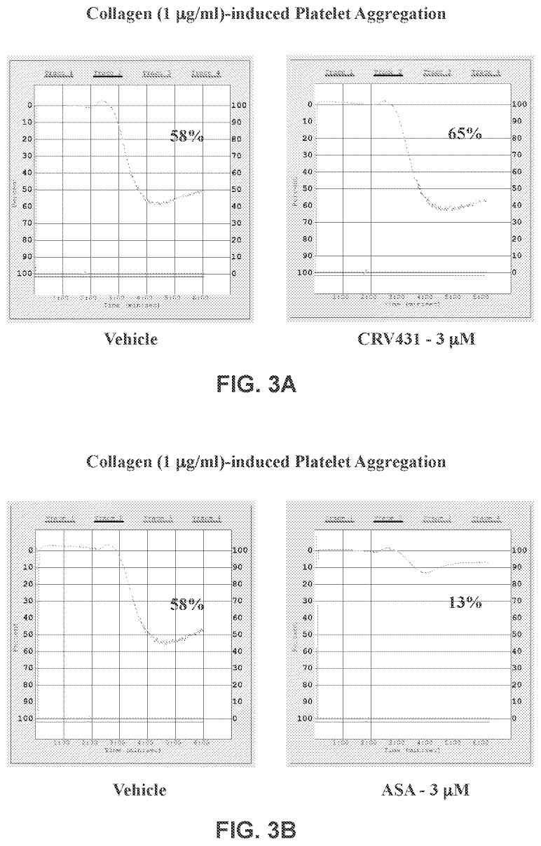

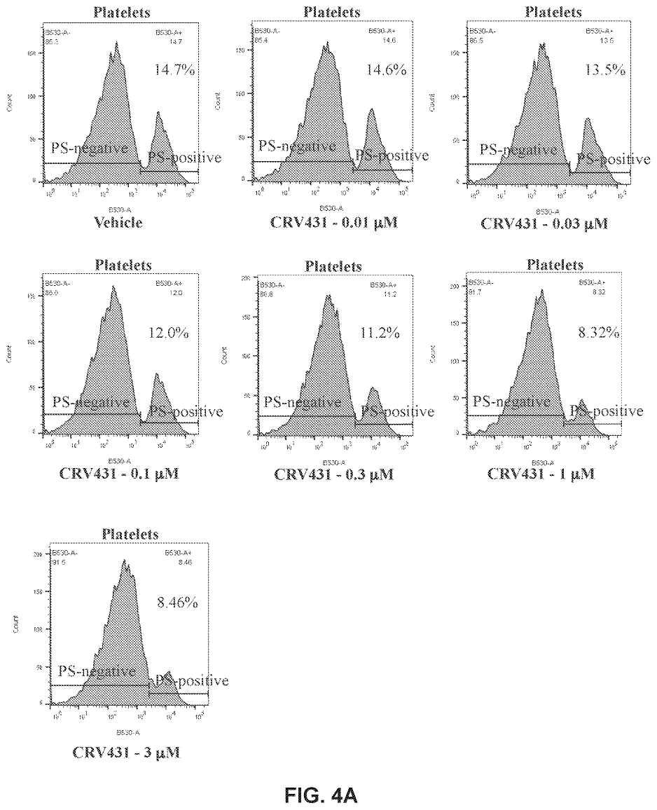

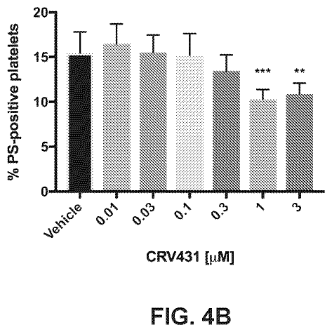

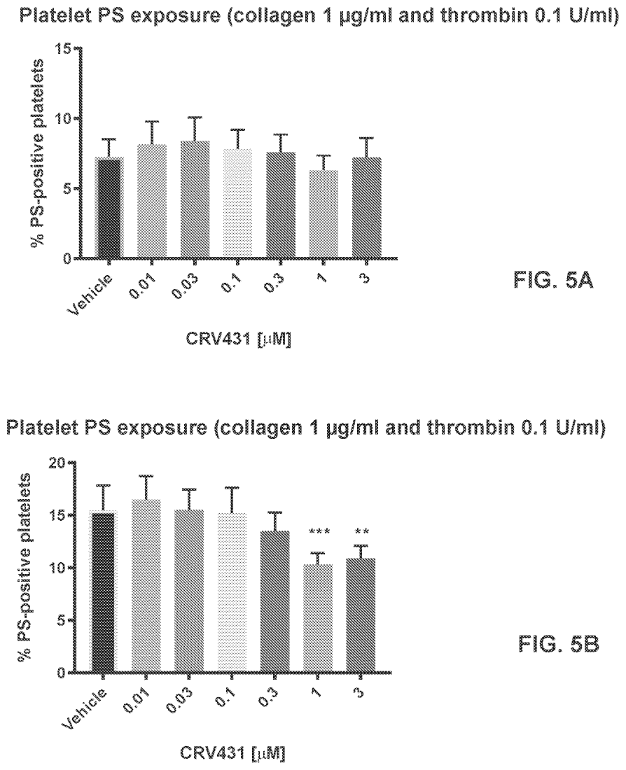

CRV431 did not inhibit platelet aggregation, induced by collagen or thrombin (FIGS. 1A-B and FIGS. 2A-B), at any of the concentrations tested (CRV431 0.01-3 μM). As a positive control the gold standard anti-platelet drug, acetylsalicylic acid, effectively inhibited aggregation (FIGS. 3A-B). However, CRV431 at 1 and 3 μM significantly inhibited platelet PS-exposure, a marker of procoagulant platelet formation, when activated by collagen and thrombin (FIGS. 4A-B). Data for platelet PS exposure in the presence of Coll 1 μg/ml-Thr 01 U/ml and Coll 10 μg/ml-Thr 0.1 U/ml are shown in FIG. 5A and FIG. 5B, respectively.

### DISCUSSION

Procoagulant platelets have been demonstrated to be elevated in thrombotic diseases. The results shown in this example indicates that CRV431 is a novel antithrombotic drug that blocks platelet-mediated coagulation via cyclophilin inhibition, but without inhibiting platelet aggregation.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the an would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, 1, or C” would include but not be limited to systems that have A alone, B alone, C alone, A and B together. A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member, Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A method for treating a thromboembolic disorder or for primary prophylaxis or secondary prophylaxis of a thromboembolic disorder, the method comprising administering to a subject in need thereof a composition comprising a therapeutically or prophylactically effective amount of CRV431, or a pharmaceutically acceptable salt, solvate, or stereoisomer thereof,

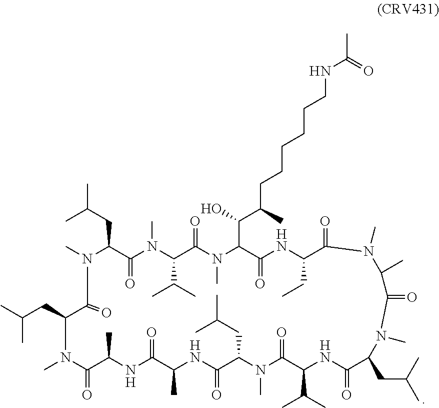 [SMILES: CC[C@@H]1NC(=O)C([C@H](O)[C@H](C)CCCCCCNC(C)=O)N(C)C(=O)[C@H](C(C)C)N(C)C(=O)[C@H](CC(C)C)N(C)C(=O)[C@H](CC(C)C)N(C)C(=O)[C@@H](C)NC(=O)[C@H](C)NC(=O)[C@H](CC(C)C)N(C)C(=O)[C@H](C(C)C)NC(=O)[C@H](CC(C)C)N(C)C(=O)C(C)N(C)C1=O]

2. The method of claim 1, wherein the thromboembolic disorder is an arterial cardiovascular thromboembolic disorder, a venous cardiovascular thromboembolic disorder, or a thromboembolic disorder in a chamber of the heart or in the peripheral circulation.

3. The method of claim 1, wherein the thromboembolic disorder is unstable angina, an acute coronary syndrome, myocardial infarction, transient ischemic attack, stroke, atherosclerosis, peripheral occlusive arterial disease, venous thrombosis, deep vein thrombosis, thrombophlebitis, arterial embolism, coronary arterial thrombosis, cerebral arterial thrombosis, cerebral embolism, kidney embolism, pulmonary embolism, or thrombosis resulting from a medical implant, device, or procedure in which blood is exposed to an artificial surface that promotes thrombosis.

4. The method of claim 1, wherein the thromboembolic disorder is stroke, myocardial infarction, unstable angina, abrupt closure following angioplasty or stent placement, thrombosis induced by a peripheral vascular surgery or a peripheral vascular disease, or a thrombotic disorder resulting from atrial fibrillation or inflammation.

5-8. (canceled)

9. The method of claim 1, wherein platelet aggregation is not inhibited in the subject.

10. The method of claim 1, wherein platelet aggregation in the subject is reduced by no more than 50%, 40%, 30%, 20%, 10%, or 5% as compared to an untreated subject.

11. The method of claim 1, wherein CRV431 selectively inhibits formation of procoagulant platelets.

12. A method of reducing or preventing thrombi formation, the method comprising administering to a subject in need thereof a composition comprising a therapeutically or prophylactically effective amount of CRV431, or a pharmaceutically acceptable salt, solvate, or stereoisomer thereof,

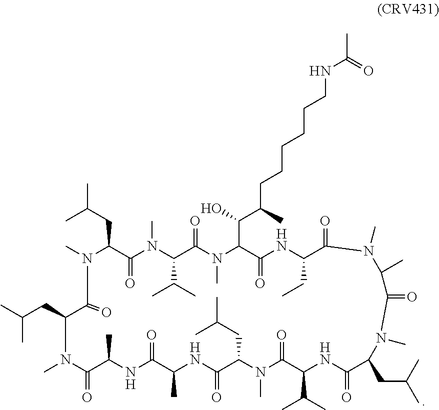 [SMILES: CC[C@@H]1NC(=O)C([C@H](O)[C@H](C)CCCCCCNC(C)=O)N(C)C(=O)[C@H](C(C)C)N(C)C(=O)[C@H](CC(C)C)N(C)C(=O)[C@H](CC(C)C)N(C)C(=O)[C@@H](C)NC(=O)[C@H](C)NC(=O)[C@H](CC(C)C)N(C)C(=O)[C@H](C(C)C)NC(=O)[C@H](CC(C)C)N(C)C(=O)C(C)N(C)C1=O]

13. A method of reducing or inhibiting procoagulant platelet formation, the method comprising administering to a subject in need thereof a composition comprising a therapeutically or prophylactically effective amount of CRV431, or a pharmaceutically acceptable salt, solvate, or stereoisomer thereof,

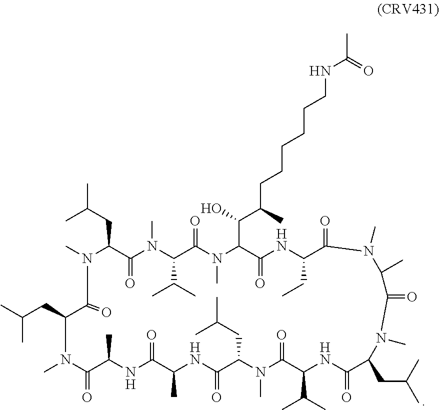 [SMILES: CC[C@@H]1NC(=O)C([C@H](O)[C@H](C)CCCCCCNC(C)=O)N(C)C(=O)[C@H](C(C)C)N(C)C(=O)[C@H](CC(C)C)N(C)C(=O)[C@H](CC(C)C)N(C)C(=O)[C@@H](C)NC(=O)[C@H](C)NC(=O)[C@H](CC(C)C)N(C)C(=O)[C@H](C(C)C)NC(=O)[C@H](CC(C)C)N(C)C(=O)C(C)N(C)C1=O]

14. The method of claim 13, comprising selectively inhibiting formation of procoagulant platelets.

15. The method of claim 13, wherein platelet aggregation is not reduced.

16. The method of claim 13, wherein the subject in need thereof is a subject suffering from or at risk of developing a prothrombotic or thrombotic condition.

17. The method of claim 16, wherein the prothrombotic or thrombotic condition is selected from the group consisting of infection, sepsis, systemic inflammatory response syndrome, multi organ failure, thrombotic thrombocytopenia purpura, hemolytic uremia syndrome, vascularization, renal failure, ischemic repercussion injury, solid organ transplant rejection, cardiovascular disease, stroke, venous thromboembolism, autoimmune disorders, sickle cell disease, inflammatory bowel disease, acute lung injury, malignancy, myocardial infarction (primary and secondary), embolic stroke, ischemic stroke, thrombotic stroke, deep vein thrombosis (DVT), thromboembolism, portal vein thrombosis, renal vein thrombosis, jugular vein thrombosis, Budd-Chiari syndrome, Paget-Schroetter disease, cerebral venous sinus thrombosis, arterial thrombosis, and arterial embolism.

18-24. (canceled)

25. The method of claim 1, wherein the subject in need thereof is receiving one or more additional therapeutic agents selected from an anti-coagulant agent, an antiplatelet agent, a fibrinolytic agent, or a combination thereof.

26. The method of claim 25, wherein the one or more additional therapeutic agents is selected from heparin, low molecular weight heparins, bivalirudin, Fondaparinux, warfarin, Acenocoumarol, Phenprocoumon, Phenindione, Abbokinase (urokinase), streptokinase, alteplase, retaplase, tenecteplase, prasugrel, aspirin, ticlopidine, clopidogrel, abciximab, eptifibatide, and tirofiba, or a combination thereof.

27-31. (canceled)

32. The method of any one of claims 1, 12, or 13, wherein the therapeutically effective or prophylactically effective amount is administered to the subject at an effective daily dose of 10 mg to 250 mg.

33-71. (canceled)
